(19) United States
(12) Patent Application Publication
(10) Pub. No.: US 2025/0389723 A1
(43) Pub. Date: Dec. 25, 2025

(54) Measurement Method

(71) Applicants: SHIMADZU CORPORATION, Kyoto-shi (JP); The University of Tokyo, Tokyo (JP)

(72) Inventors: Kengo AOKI, Kyoto-shi (JP); Ryutaro ODA, Kyoto-shi (JP); Seiichi OHTA, Tokyo (JP); Hiroki TSUCHIYA, Tokyo (JP); Noriko NAKAMURA, Tokyo (JP)

(21) Appl. No.: 19/102,773

(22) PCT Filed: Jul. 24, 2023
(86) PCT No.: PCT/JP2023/026962

§ 371 Date: Feb. 10, 2025

(30) Foreign Application Priority Data
Aug. 10, 2022 (JP) 2022-127552
Aug. 30, 2022 (JP) 2022-137093

(51) Int. Cl. G01N 33/58; G01N 33/53; G01N 33/552

(52) U.S. Cl. CPC G01N 33/582; G01N 33/5308; G01N 33/552

(57) ABSTRACT

Labels (2a, 2b) of the first label group and labels (2c, 2d) of the second label group are different from each other in label property. In each particle group, a plurality of particle subgroups (a to d) are different from each other in particle size. A measurement method further comprises: mixing a specimen, particles (1a to 1d), and labels (2a to 2d); allowing biomolecules to specifically bind to the labels (2a to 2d) and to the labels (2a to 2d); separating the particles based on particle sizes; detecting label properties of the labels; determining types of the biomolecules bound to the particles based on the particle size and the label property.

### TECHNICAL FIELD

The present invention relates to a measurement method, and more specifically, it relates to a measurement method to measure a biomolecule.

### BACKGROUND ART

Conventionally, there are objective assessment methods applicable to disease diagnosis and/or therapeutic effect determination, which include methods to measure biomolecules (which are referred to as biomarkers) in a living body. Promising among these is a method that, on the premise that a disease occurs as a combined total of actions of multiple biomolecules, makes a diagnosis and/or a therapeutic effect determination based on results of measurement of multiple types of biomarkers.

As a method for simultaneously detecting multiple types of biomarkers, a method called xMAP (registered trademark) is known. As one xMAP technique, Japanese National Patent Publication No. 2012-533052 (PTL 1) discloses a Luminex (registered trademark) technique as a method for detecting biomarkers. xMAP is a technique that uses, as a library for distinguishing biomarker types, beads containing fluorescent dyes of two different colors mixed in certain concentrations to exhibit certain fluorescence spectra. In xMAP, such beads with different fluorescence spectra are configured to bind to different types of biomarkers.

### CITATION LIST

### Patent Literature

PTL 1: Japanese National Patent Publication No. 2012-533052

### SUMMARY OF INVENTION

### Technical Problem

However, in xMAP, it is necessary to differentiate among the beads based solely on the fluorescence spectra, so it is difficult to distinguish among beads that exhibit similar fluorescence spectra. As a result, there has been a limit, in principle, on the number of types of biomarkers that can be detected simultaneously. Hence, there has been a demand for a technique other than xMAP that allows for simultaneously detecting multiple types of biomarkers.

The present disclosure has been devised to overcome the above-described shortcoming, and it aims at providing a technique that enables simultaneous measurement of multiple types of biomarkers.

### Solution To Problem

A first aspect of the present disclosure is a measurement method to measure a biomolecule contained in a specimen derived from a biological specimen, the measurement method comprising: preparing particles belonging to a first particle group and particles belonging to a second particle group; and preparing labels belonging to a first label group corresponding to the first particle group and labels belonging to a second label group corresponding to the second particle group. The labels of the first label group and the labels of the second label group are different from each other in label property. The particle of the first particle group and the label of the first label group bind to each other via a biomolecule. The particle of the second particle group and the label of the second label group bind to each other via a biomolecule. Each of the first particle group and the second particle group includes a plurality of particle subgroups. In each of the first particle group and the second particle group, the plurality of particle subgroups are different from each other in particle size. Particles of the plurality of particle subgroups in the first particle group and particles of the plurality of particle subgroups in the second particle group have first binding portions, and the first binding portions for different particle subgroups are capable of specifically binding to different types of biomolecules. The measurement method further comprises: mixing together the specimen, the particles of the first particle group and the particles of the second particle group, and the labels of the first label group and the labels of the second label group; allowing biomolecules to specifically bind to the particles of the first particle group and the particles of the second particle group as well as to the labels of the first label group and the labels of the second label group; separating among the particles of the first particle group and among the particles of the second particle group based on particle sizes; detecting label properties of the labels bound respectively via the biomolecules to the particles of the first particle group and the particles of the second particle group thus separated based on the particle sizes; and based on the particle sizes and the label properties, determining types of the biomolecules bound to the particles of the first particle group and the particles of the second particle group.

A second aspect of the present disclosure is a measurement method to measure a biomolecule contained in a specimen derived from a biological specimen, the measurement method comprising: preparing particles belonging to a third particle group and particles belonging to a fourth particle group; and preparing labels belonging to a third label group corresponding to the third particle group and labels belonging to a fourth label group corresponding to the fourth particle group. The particles belonging to the third particle group and the particles of the fourth particle group are different from each other in particle property. The particle of the third particle group and the label of the third label group bind to each other via a biomolecule. The particle of the fourth particle group and the label of the fourth label group bind to each other via a biomolecule. Each of the third particle group and the fourth particle group includes a plurality of particle subgroups. In each of the third particle group and the fourth particle group, the plurality of particle subgroups are different from each other in particle size. Particles of the plurality of particle subgroups in the third particle group and particles of the plurality of particle subgroups in the fourth particle group have third binding portions, and the third binding portions for different particle subgroups are capable of specifically binding to different types of biomolecules. The measurement method further comprises: mixing together the specimen, the particles of the third particle group and the particles of the fourth particle group, and the labels of the third label group and the labels of the fourth label group; allowing biomolecules to specifically bind to the particles of the third particle group and the particles of the fourth particle group as well as to the labels of the third label group and the labels of the fourth label group; separating among the particles of the third particle group and among the particles of the fourth particle group based on particle sizes; detecting particle properties of the particles of the third particle group and the particles of the fourth particle group thus separated based on the particle sizes, as well as label properties of the labels bound via the biomolecules to the particles; and based on the particle sizes, the particle properties, and the label properties, determining types of the biomolecules bound to the particles of the third particle group and the particles of the fourth particle group.

A third aspect of the present disclosure is a measurement method to measure a biomolecule contained in a specimen derived from a biological specimen, the measurement method comprising: preparing particles belonging to a first particle group; and preparing labels belonging to a first label group corresponding to the first particle group. The particle of the first particle group and the label of the first label group bind to each other via a biomolecule. The first particle group includes a plurality of particle subgroups. In the first particle group, the plurality of particle subgroups are different from each other in particle size. Particles of the plurality of particle subgroups in the first particle group have first binding portions, and the first binding portions for different particle subgroups are capable of specifically binding to different types of biomolecules. The measurement method further comprises: mixing the specimen, the particles of the first particle group, and the labels of the first label group to allow biomolecules to specifically bind to the particles of the first particle group and the labels of the first label group; separating the particles of the first particle group based on particle sizes; detecting label properties of the labels bound respectively via the biomolecules to the particles of the first particle group thus separated based on the particle sizes; and based on the particle sizes and the label properties, determining types of the biomolecules bound to the particles of the first particle group.

### Advantageous Effects Of Invention

A control apparatus according to the present disclosure makes it possible to provide a technique for simultaneously measuring multiple types of biomolecules.

### BRIEF DESCRIPTION OF DRAWINGS

FIG. 1 illustrates the entire configuration of a measurement system according to the present embodiment.

FIG. 2 is a view for explaining the structure of a particle and a label.

FIG. 3 is a view for explaining particle groups and label groups.

FIG. 4 is a view for explaining a method for binding a particle, a biomarker, and a label together.

FIG. 5 is a view showing results of separation based on the particle size.

FIG. 6 is a view showing results of label property detection.

FIG. 7 is a flowchart illustrating a measurement process according to the present embodiment.

FIG. 8 is a flowchart illustrating a measurement process according to Variation 1.

FIG. 9 is a view for explaining a specific example of correction with the use of a first correction particle.

FIG. 10 is a view for explaining a specific example of correction with the use of a second correction particle.

FIG. 11 is a view for explaining the structure of a particle and a label according to Variation 2.

FIG. 12 is a view for explaining particle groups and label groups according to Variation 2.

FIG. 13 is a flowchart illustrating a measurement process according to Variation 2.

### DESCRIPTION OF EMBODIMENTS

In the following, a detailed description will be given of embodiments of the present invention, with reference to drawings. The same or corresponding portions in the drawings are denoted by the same reference characters, and the description thereof is not repeated.

### 1. Configuration of Measurement System

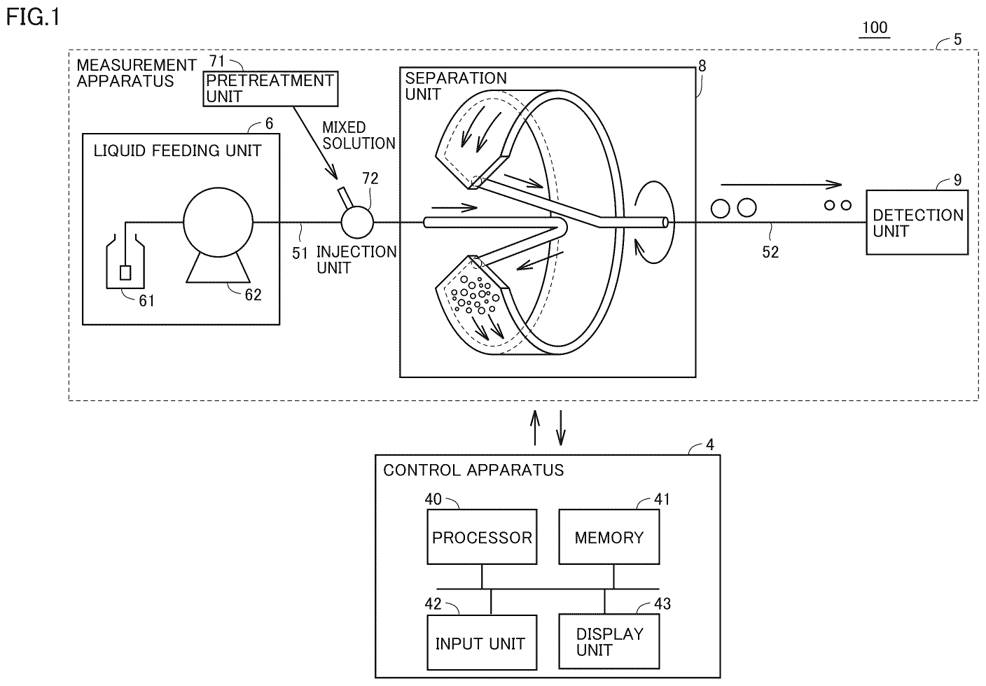

FIG. 1 is a view for explaining the outline of a measurement system 100 according to an embodiment of the present invention.

Referring to FIG. 1, measurement system 100 includes a control apparatus 4 and a measurement apparatus 5.

Measurement apparatus 5 is an apparatus for measuring biomarkers. Measurement apparatus 5 includes a liquid feeding unit 6, a pretreatment unit 71, an injection unit 72, a separation unit 8, channels 51, 52, and a detection unit 9.

In measurement apparatus 5, at downstream of liquid feeding unit 6, channel 51 is connected. Liquid feeding unit 6 feeds a carrier (a mobile phase) to channel 51. Liquid feeding unit 6 includes a vessel 61 to store the carrier and a liquid-feeding pump 62 for sucking the carrier from vessel 61.

Channel 51 is a channel connecting liquid feeding unit 6 with separation unit 8. On channel 51, injection unit 72 is provided.

Injection unit 72 is a unit for injecting a mixed solution into the carrier inside the channel 51. The mixed solution is a solution produced in pretreatment unit 71 from a specimen. The method for producing the mixed solution by pretreatment unit 71 will be described later. The mixed solution includes particles for measuring biomarkers. The particles include a particle to which a biomarker is bound and a particle to which a biomarker is not bound, and in the description of FIG. 1, they are collectively referred to as “particles”. To each biomarker, a label having a certain label property (fluorescence, for example) is bound. Injection unit 72 may be an autosampler, or may be an inlet through which a user can manually inject the mixed solution into channel 51, for example.

Separation unit 8 separates the particles mixed with the carrier (hereinafter also referred to as “the particles included in the carrier”), based on the particle size. Generally, separating particles based on the size is referred to as “classification”. In an example, separation unit 8 is a classification apparatus that uses a centrifugal field flow fraction (FFF) method or an asymmetrical flow field flow fraction (AF4) method, for example, each of which is a type of FFF methods.

The centrifugal FFF method is a method to let large particles become sedimented by centrifugal force to classify the particles based on the difference in centrifugal force and diffusion coefficient. With the centrifugal FFF method, it is possible to classify particles based on the particle mass and the particle size. The size of particles can be expressed as the diameter, volume, and the like of the particles, for example, when the particles are substantially spherical. The centrifugal FFF method has a relatively high size-resolving power, so it is capable of distinguishing among many types of biomarkers, which is an advantage. In addition, as compared to an AF4 method, the centrifugal FFF method can perform classification with less errors, so it can produce classification results with high reproducibility. Because of this, it is not necessary to take into account the influences of classification errors on the results of label property measurement performed at detection unit 9. As a result, accuracy of label property measurement is enhanced, and accuracy of biomarker quantification is enhanced.

The AF4 method is a method to classify particles based on the difference in the speed of the particles moving in a laminar flow that is generated by a force field vertical to the direction of the movement. With the centrifugal FFF method, it is possible to classify particles based on the particle size. The size of particles can be expressed as the diameter, volume, and the like of the particles, for example, when the particles are substantially spherical. On the other hand, the AF4 method can also classify small, light-weight particles, which is an advantage. This makes it possible to use small particles for biomarker detection, thereby allowing for distinguishing among many types of biomarkers.

Separation unit 8 may be a classification apparatus that uses size exclusion chromatography. In size exclusion chromatography, a solution that includes particles is made to flow through a column that has many pores. Then, due to the phenomenon where smaller particles enter the pores and become eluted late, the particles are classified. With size exclusion chromatography, it is possible to classify particles based on the particle size. The size of particles can be expressed as the diameter, volume, and the like of the particles, for example, when the particles are substantially spherical. A classification apparatus that uses size exclusion chromatography can be configured at low cost, as compared to a classification apparatus that uses a field flow fraction (FFF) method.

Channel 52 is a channel connecting separation unit 8 with detection unit 9. The particle-including carrier discharged from separation unit 8 flows through channel 52 into detection unit 9.

Detection unit 9 detects the label property of a label bound via a biomarker to each particle separated based on the particle size. In the present embodiment, the label property is not particularly limited as long as it is a property that allows for differentiating among different types of labels 2 corresponding to different types of biomarkers 3. In an example, the label property is fluorescence. In this case, detection unit 9 includes a plurality of fluorescence detectors or multi-wavelength fluorescence detectors, for example. A multi-wavelength fluorescence detector is a detector that is capable of performing measurement at a plurality of excitation wavelengths and fluorescence wavelengths at the same time. When a plurality of fluorescence detectors are used as detection unit 9, highly sensitive detection of fluorescence is made possible, so accuracy of label property measurement is enhanced, and accuracy of biomarker quantification is enhanced. When a multi-wavelength fluorescence detector is used as detection unit 9, the number of detectors can be reduced, and thereby the cost for detection unit 9 can be reduced. In another example, detection unit 9 is a radiation measurement instrument or an absorption spectrometer.

At detection unit 9, label properties of the plurality of particles may be detected on a one-by-one basis, or label properties of the particles may be detected all at once. For example, when detection unit 9 is configured in such a way that only one or less particle can be present at one time at a label-substance-measurement position, the label properties of the particles are detected on a one-by-one basis. Examples of this configuration include when a label-substance-measurement position of the channel is narrow and only one particle can pass the position at one time, and when the concentration of particles in the carrier is low enough to allow only one molecule to be present at the measurement position.

In contrast, when it is configured in such a way that a plurality of particles can be present at one time at a label-substance-measurement position, a detected value corresponding to a combined total of the label properties of the plurality of particles is detected. In this case, by calculating, from the detected value corresponding to the combined total, the number of detected particles with respective label properties, it is possible to determine the types of the corresponding biomarkers and the numbers of them.

Control apparatus 4 controls measurement apparatus 5, and analyzes the detection results from detection unit 9. Control apparatus 4 is typically a computer, and can be implemented as a dedicated computer or a general-purpose personal computer.

Control apparatus 4 comprises a processor 40, a memory 41, an input unit 42, and a display unit 43.

Processor 40 includes a central processing unit (CPU), for example. Processor 40 decompresses a program stored in memory 41, to execute it on an RAM and the like.

Memory 41 includes a read only memory (ROM), a random access memory (RAM), and a non-volatile memory, for example. A program stored in the ROM is a program that describes process steps for measurement system 100. The non-volatile memory stores detection results that are transmitted from detection unit 9, in the form of data file. Instead of or in addition to the non-volatile memory, memory 41 may include a hard disk drive (HHD) and/or a solid state drive (SSD).

Input unit 42 is a unit at which a user can input commands for measurement system 100. For example, input unit 42 includes a keyboard, and a pointing device such as a mouse.

Display unit 43 includes a liquid crystal display and the like. Display unit 43 displays the detection results from detection unit 9, as well as the results of analyzing them.

Control apparatus 4 may be configured with a plurality of computers. Part of or all of the above-mentioned functions of control apparatus 4 may be provided at an electronic computer, a server, and the like physically apart from measurement apparatus 5. For example, control apparatus 4 may include a system controller which is a dedicated computer, as well as a general-purpose personal computer connected to the system controller via a network.

### 2. Conventional Biomarker Measurement Method

For disease diagnosis and/or therapeutic effect determination, it is necessary to objectively assess the presence or absence of a disease, the rate of its progress, the intensity of symptoms, and the like. For such objective assessment of a disease, a method capable of measuring a biomarker in a living body to use it as an index is useful. Particularly promising is a method that recognizes a disease as a combined total of interactions between many biomolecules and makes a diagnosis based on a set of measured values of many types of biomarkers.

For conventional methods such as enzyme-linked immuno-sorbent assay (ELISA) which capture biomarkers in a type-specific manner and then label respective types of captured biomarkers to detect the labels corresponding to the respective types of biomarkers, it is difficult to measure many types of biomarkers, in terms of testing time and cost.

On the other hand, as a method for simultaneously measuring multiple types of biomarkers, xMAP (registered trademark) is known, and xMAP systems such as Luminex (registered trademark) are commercially available. Herein, “simultaneously measuring multiple types of biomarkers” refers to “simultaneously labeling multiple types of biomarkers and simultaneously detecting them”. “Simultaneously” refers to “in one step”, for example. In xMAP, microbeads are stained with two fluorescent dyes of different colors mixed in various concentrations. Then, the fluorescence spectra reflecting the patterns of combinations of the fluorescent dye concentrations in the beads are used as discrimination codes.

In an xMAP-based biomarker measurement experiment, firstly, fluorescence-emitting label substances are made to bind to biomarkers that are specifically bound to beads. Then, the fluorescence spectra of the beads are measured to measure the multiple types of biomarkers.

Here, when differentiating the beads based solely on the fluorescence spectra of the beads, it is difficult to distinguish among beads with similar fluorescence spectra. Due to this limitation, the number of beads that can be simultaneously detected, more specifically, the number of beads whose fluorescence spectra are different from each other to the extent that they can be distinguished from each other is relatively small. The expression “whose fluorescence spectra are different from each other to the extent that they can be distinguished from each other” refers to, for example, a state where the positions on the horizontal axis (wavelength) corresponding to the peaks of the fluorescence spectra are located apart from each other to the extent that they can be differentiated from each other. For example, like in xMAP, when beads are differentiated from each other based on the fluorescence spectra reflecting the ratio between two dyes with different colors, it is practically conceivable that the number of types of beads that can be differentiated is about thirty. Therefore, in principle, it is difficult to increase the number of types of biomarkers that can be simultaneously detected, beyond this number.

On the other hand, from the viewpoint where many biomolecules can be involved with a disease, it is desirable to obtain measured values of more types of biomarkers (a hundred types, for example) for making a disease diagnosis and/or a therapeutic effect determination. Because of this, in the clinical and research settings, there is a demand for a technique that allows for simultaneously measuring many types of biomarkers.

In light of these circumstances, with a biomarker measurement method according to the present embodiment in which differentiation is performed not only by using the label property such as fluorescence but also by using particles with different particle sizes, it is made possible to increase the number of types of biomarkers that can be discriminated from each other. As a result, it is made possible to simultaneously measure many types of biomarkers.

### 3. Measurement Method According to Embodiment

### (3-1. Structure of Particles and Labels)

Firstly, the structure of particles and labels used in the measurement method according to the present embodiment will be described.

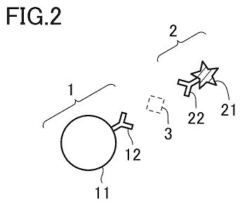

FIG. 2 is a view for explaining the structure of a particle and a label.

FIG. 2 illustrates biomarker 3, as well as a particle 1 and label 2 that bind to biomarker 3.

Herein, biomarker 3 refers to a biomolecule, a measurement target of the measurement method according to the present embodiment, which serves as an index for quantitatively understanding biological changes in a living body such as the presence or absence of a disease, the rate of its progress, and the efficacy of drugs. In an example, the biomarker is a protein. The biomarker may be at least one of a nucleic acid and a metabolite. The nucleic acid may include deoxyribonucleic acid (DNA), messenger ribonucleic acid (RNA), long-chain noncoding RNA, or microRNA.

Each particle 1 includes a particle body 11, as well as a first binding portion 12 that is capable of specifically binding to biomarker 3.

Each particle body 11 is typically a sphere having a certain diameter, but the shape may have a certain range of variations due to the production process and the like. The certain range is, for example, a range within which classification can be performed by separation unit 8 without problems. Like particle body 11 or particle 1, any spherical object usable in measurement of biological specimens, or any object that is obtained by adding certain modifications to the spherical object, is also referred to as a “bead” by a person skilled in the art. The material for forming particle body 11 includes, for example, at least one of an inorganic material such as gold or silica (silicon dioxide) and a resin material such as polystyrene. In the case when particle body 11 is a gold particle made of gold, the diameter of particle body 11 is preferably a certain value from 5 to 500 nm. When particle body 11 is a silica particle made of silica, the diameter of particle body 11 is preferably a certain value from 10 to 1000 nm.

Hereinafter, the above-described gold particles and the above-described silica particles with nanoscale sizes are also referred to as “gold nanoparticles” and “silica particles”, respectively. In the following, advantages of using gold nanoparticles and silica nanoparticles as particle bodies 11 will be described.

Gold nanoparticles are characteristically stable and less prone to degradation. Because of this, during storage or measurement, over-time degradation or chemical-or impact-induced degradation tends not to occur. As a result, chances of particle size changes to occur during storage or measurement are slim, which can result in enhanced reliability of analysis. Furthermore, gold nanoparticles are characteristically easy to perform size control at the time of production. Because of this, the particles can be made with small variations in size distribution. This allows for widening the range of size variations that can be simultaneously separated from each other. As a result, when gold nanoparticles are used as particle bodies 11, it is possible to increase the number of types of biomarkers 3 that can be simultaneously detected.

As compared to gold nanoparticles, silica particles scatter less light (which can affect detection) at the particle surfaces, because the refractive index of silica is closer to that of water than that of gold is. Furthermore, unlike gold, silica does not exhibit a high absorbance at any particular wavelength. For this reason, when silica nanoparticles are used as particle bodies 11, accuracy of measurement in terms of fluorescence intensity and absorbance is high. As a result, accuracy of quantification of biomarker 3 is high.

An advantage of using resin-material-based nanoparticles as particle bodies 11 is that there are commercially available resin-material-based nanoparticles that are traceable and highly reliable, so it is possible to perform the measurement relatively easily with accuracy.

In an example, particle body 11 is a gold spherical particle coated with silica on the surface. By using particle body 11 of this type, it is possible to simultaneously detect many types of biomarkers 3 and accurately measure the amount of the biomarkers.

Preferably, the difference between the refractive index of particle 1 and the refractive index of the carrier (hereinafter also referred to as “the refractive index difference”) does not exceed a certain numerical value. More specifically, it is preferable that the refractive index difference between particle body 11 and the carrier do not exceed a certain numerical value. The refractive index difference is not limited to the above-mentioned range, but it is preferably 0.1 or less, more preferably 0.05 or less. In the following, the reason why the refractive index difference preferably does not exceed the certain numerical value will be described in detail.

When there is a refractive index difference between particle 1 and the carrier, at the time of excitation light irradiation from a fluorescence detector, the light can be scattered by the particles. In this case, even when label 2 is not present, the scattered light can enter the fluorescence detector, and the fluorescence detector can detect a signal. For accurately detecting the fluorescence emitted by label 2, it is preferable to set the lower detection limit for the fluorescence detector not to include a detection signal attributable to scattered light. It is also preferable to set the lower detection limit low enough to ensure a sufficient level of sensitivity for diagnosis. Accordingly, it is preferable to set the refractive index difference between the particle and the carrier not to exceed the certain numerical value so that the amount of scattering light attributable to particle 1 does not exceed a certain amount. As a result, the lower limit to fluorescence detection can be set low, and detection sensitivity can be enhanced.

In contrast, when the refractive index difference between particle 1 and the carrier exceeds the certain value, more light is scattered by particles 1 at the time of excitation light irradiation. In this case, if the lower detection limit is raised in accordance with the amount of scattered light, it may become impossible to detect label 2 with sufficient sensitivity for diagnosis.

A first method for lowering the refractive index difference between particle 1 and the carrier is to use a carrier the refractive index of which is close to the refractive index of particles 1. A second method is to use particles 1 the refractive index of which is close to the refractive index of the carrier. A third method is to bring the refractive index of particles 1 and the refractive index of the carrier close to each other.

In an example, the refractive index of particles 1 is about 1.5 and the refractive index of the carrier is 1.33. Particles 1 of this type are silica particles (refractive index, 1.46), for example, and the carrier of this type is water (refractive index, 1.33), for example. In this example, the first method may be replacing the carrier having a refractive index of 1.33 by a carrier having a refractive index of 1.5. The second method may be replacing particles 1 having a refractive index of 1.5 by particles 1 having a refractive index of 1.33. The third method may be replacing the carrier having a refractive index of 1.33 by a carrier having a refractive index of 1.4, and replacing particles 1 having a refractive index of 1.5 by particles 1 having a refractive index of 1.4. Such replacement of particles 1 and the carrier includes replacing the original set of particles 1 and the carrier by a new set of particles 1 and a carrier, as well as modifying the original set of particles 1 and the carrier to change the refractive indices. Such change includes coating the original particles 1, re-preparing the carrier by adding a new component thereto, and the like, for example.

The extent of the above-described erroneous detection of scattered light attributable to particles 1 can be affected not only by the refractive index difference but also by the excitation wavelength of the fluorescence detector, the fluorescence wavelength, the bandwidth of the excitation wavelength, the bandwidth of the fluorescence wavelength, and the like. For example, the closer the excitation wavelength and the fluorescence wavelength are to each other, the higher the chances of erroneous detection are. Therefore, it is preferable to design particles 1 and labels 2 and also to set the fluorescence detector, including the excitation wavelength, the fluorescence wavelength, the bandwidth of the excitation wavelength, the bandwidth of the fluorescence wavelength, and the like, so that erroneous detection is less likely to occur and a sufficient level of detection sensitivity for diagnosis is ensured.

First binding portion 12 is bound to particle body 11. First binding portion 12 is a binding site on particle 1 for specifically binding to a certain type of biomarker 3. Due to this, particle 1 has binding specificity to biomarker 3. In an example, when biomarker 3 is a certain type of protein, first binding portion 12 typically includes an antibody capable of binding to the certain type of protein. In this case, the antibody and the certain type of protein have respective structures that are capable of specifically binding to each other via a hydrogen bond and/or a force such as Coulomb force and the van der Waals forces. In another example, when biomarker 3 is a nucleic acid having a certain sequence, first binding portion 12 includes a nucleic acid that has a sequence complementary to a first sequence, which is a part of the certain sequence. The nucleic acid includes a microRNA. As for the complementary sequence, between the first sequence and the nucleic acid having the complementary sequence, adenine-uracil and guanine-cytosine hydrogen bonds are formed, for example. In a yet another example, when biomarker 3 is a certain type of metabolite, first binding portion 12 typically includes a molecule that is capable of specifically binding to a part of the certain type of metabolite.

Label 2 includes a labeling portion 21, as well as a second binding portion 22 that is capable of specifically binding to biomarker 3.

Labeling portion 21 is a portion that includes a substance for labeling particle 1 to which biomarker 3 is bound (hereinafter the substance is also referred to as “a label substance”). The label substance exhibits a certain label property. It is configured so that the respective label properties of labeling portions 21 of labels 2 corresponding to different types of biomarkers 3 can be differentiated from each other. As a result, the types of biomarkers 3 can be distinguished from each other based on the difference in the label properties of the label substances.

In an example, the label substance is a fluorescent substance and the label property is fluorescence. The fluorescence may be detected as a fluorescence spectrum that indicates the relationship between wavelength and fluorescence intensity, or as its pattern (the shape of the graph), or, alternatively, only the intensity thereof at a certain wavelength may be detected. The fluorescent substance is usually one type of fluorescent dye, but it may be two or more types of fluorescent dyes mixed together. As the fluorescent dye, a dye that is generally used in fluorescence-activated cell sorting (FACS) is used, for example. As a more specific example, a dye that is generally used for labeling may be used, such as those belonging to the Alexa Fluor (registered trademark) family with different wavelengths, rhodamine, and propidium iodide (PI).

The fluorescence spectra for respective types of labeling portions 21, which correspond to respective types of biomarkers 3, have fluorescence wavelengths characteristic of the respective fluorescence spectra, for example. A fluorescence wavelength characteristic of a fluorescence spectrum of a certain type of labeling portion 21 is, for example, a fluorescence wavelength at which the fluorescence intensity of the certain type of labeling portion 21 is high but the fluorescence intensity of other types of labeling portions 21 is very low. In this case, by referring to the fluorescence intensity at the wavelength, it is possible to determine the presence or absence of the certain type of labeling portion 21.

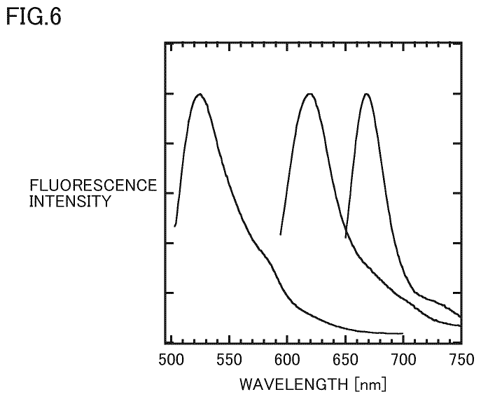

More specifically, the fluorescence spectra for respective types of labeling portions 21, which correspond to respective types of biomarkers 3, are preferably configured so that their peaks substantially do not overlap with each other (see FIG. 6). Specifically, labeling portions 21 are configured so that the wavelengths for the peak tops for respective types of labeling portions 21 are sufficiently apart from each other. Moreover, at a wavelength for the peak top for a certain type of labeling portion 21, fluorescence intensity for other types of labeling portions 21 is substantially not detected. In this case, by referring to the fluorescence intensities at the wavelengths for the peak tops for respective types of labeling portions 21, it is possible to differentiate among the respective types of labeling portions 21.

In another example, the label substance is a radioisotope and the label property is the amount of radiation. The amount of radiation may be detected as a radiation spectrum that indicates the relationship between the wavelength and the amount of radiation, or as its pattern (the shape of the graph), or, alternatively, only the radiation intensity at a certain wavelength may be detected.

In a yet another example, the label substance is a substance that exhibits a certain absorbance and the label property is absorbance. The absorbance may be detected as an absorption spectrum that indicates the relationship between the wavelength and the amount of absorbance, or as its pattern (the shape of the graph), or, alternatively, only the absorbance at a certain wavelength may be detected. When a substance that exhibits a certain absorbance is used as the label substance, because gold highly absorbs purple and blue lights, it is preferable that the surface of particle body 11 be formed with a substance other than gold (silica, for example) for avoiding influence of particle body 11 on the detection of the label substance. The label substance may be a substance that exhibits a color of a wavelength that can be recognized by the naked eye, and the label property may be the color. It should be noted that the label substance and the label property are not limited to the above-mentioned examples, and they are simply required to allow for differentiating among labeling portions 21 corresponding to respective types of biomarkers 3.

Second binding portion 22 is bound to labeling portion 21. Second binding portion 22 is a binding site on label 2 for specifically binding to a certain type of biomarker 3. Due to this, label 2 has binding specificity to biomarker 3. It should be noted that second binding portion 22 binds to a site on biomarker 3 that is different from the binding site for first binding portion 12. Accordingly, label 2 can bind to biomarker 3 to which particle 1 is bound. In an example, when biomarker 3 is a certain type of protein, second binding portion 22 typically includes an antibody capable of binding to the certain type of protein. In another example, when biomarker 3 is a nucleic acid having a certain sequence, second binding portion 22 typically includes a nucleic acid that has a sequence complementary to a second sequence, which is a part of the certain sequence. It should be noted that the second sequence is a different sequence from the first sequence to which first binding portion 12 binds. In a yet another example, when biomarker 3 is a certain type of metabolite, second binding portion 22 typically binds to the certain type of metabolite in a specific manner at a site that is different from the site to which first binding portion 12 binds. In this way, the measurement method according to the present embodiment can measure biomarkers 3 belonging to any of the categories of proteins, nucleic acids, and metabolites.

In an example, second binding portion 22, which was made to bind to labeling portion 21 in advance, specifically binds to biomarker 3. Alternatively, second binding portion 22 may be configured so that it specifically binds to biomarker 3 before binding to labeling portion 21 and then specifically binds to labeling portion 21.

The manner of binding between second binding portion 22 and labeling portion 21 is not particularly limited, and, for example, it may be amide bond formation via carbodiimide reactions, avidin-biotin interaction, or cyclooctyne-azide click chemistry.

### (3-2. Size of Particle, Biomarker, and Label)

In the following, the relationship in size between particle body 11, first binding portion 12, biomarker 3, and label 2 will be described more.

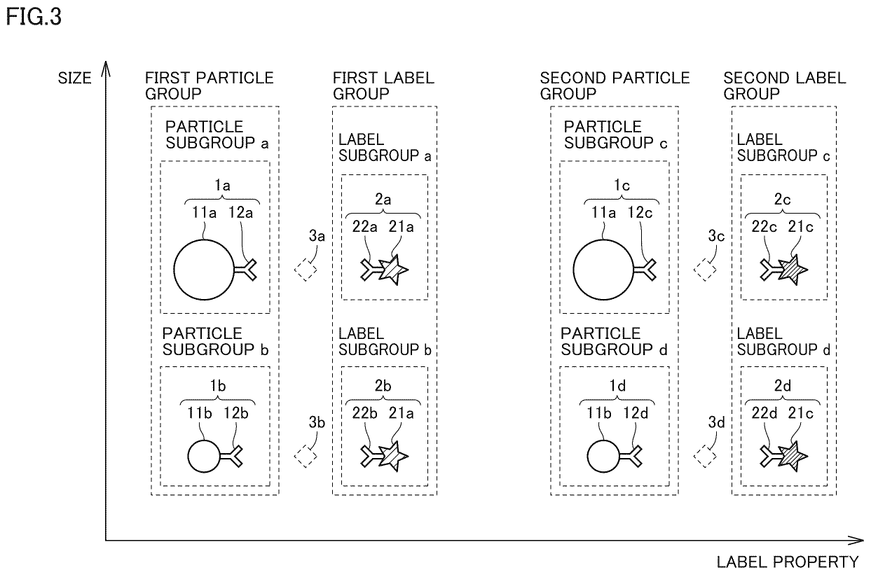

In the measurement method according to the present embodiment, as described in FIG. 3 in detail, particle bodies 11 with different sizes are used. More specifically, a plurality of particle bodies 11 with a plurality of sizes that can be classified at separation unit 8 are used. Herein, the “size” of an object refers to a value that indicates the size and/or mass of the object. For example, the size of an object can be expressed as the largest outer dimension (the diameter in the case of a sphere) and/or the volume. In an example, regardless of the sizes of particle bodies 11, the compositions of particle bodies 11 are substantially equivalent to each other, and the specific gravities of particle bodies 11 are also substantially equivalent to each other. In this case, the diameters, the volumes, and/or the masses of particle bodies 11 correlate with each other.

As for a plurality of particle bodies 11 with different sizes, it is preferable to configure the difference in size to be sufficiently larger than the size of first binding portion 12, the size of biomarker 3, and the size of label 2. The size here refers to, for example, a physical quantity for the size that affects the classification at separation unit 8 (for example, the largest outer dimension, volume, and/or mass). With this configuration, regardless of the type of first binding portion 12, biomarker 3, and label 2 bound to particle body 11, it is possible to perform classification at separation unit 8 based on the sizes of particle bodies 11.

Similarly, as for a plurality of particle bodies 11 with different sizes, it is preferable to configure the size of the smallest particle body 11 to be sufficiently larger than the size of first binding portion 12, the size of biomarker 3, and the size of label 2. With this configuration, regardless of the type of first binding portion 12, biomarker 3, and label 2 bound to particle body 11, it is possible to perform classification at separation unit 8 based on the sizes of particle bodies 11. It is also possible to perform accurate classification between a free-form label 2 included in the mixed solution and the smallest particle body 11. Thereby, it is possible to avoid erroneous detection of a free-form label 2 as a smallest labeled particle body 11.

As a result, the size of a particle 1 that is composed of a particle body 11 of a certain size and a first binding portion 12 bound thereto is approximately equivalent to the particle body 11. Also, the size of a complex composed of a particle 1 and a biomarker 3 bound thereto (hereinafter also referred to as “a biomarker complex”), as well as the size of a complex composed of the above-mentioned complex and a label 2 bound thereto (hereinafter also referred to as “a label complex”), are not substantially different from the size of the particle body 11. In other words, the sizes of particle body 11 as a base, particle 1, the biomarker complex, and the label complex are approximately equivalent to each other. In light of these circumstances, herein, the size of particle body 11 and the size of particle 1 are collectively referred to as “the particle size”. In addition, when biomarker 3 is bound to particle 1 to form a biomarker complex, the size of the biomarker complex is also referred to as “the particle size”. Furthermore, when biomarker 3 and label 2 are bound to particle 1 to form a label complex, the size of the label complex is also referred to as “the particle size”.

Herein, the expression that reads “different from each other in particle size” refers to, for example, a state where the particle size distributions of different particle groups (for example, different particle subgroups) do not overlap with each other. Alternatively, when some extent of overlapping is tolerated, the above-mentioned expression may refer to a state where the average particle sizes are different from each other. Anyway, the particle size may be selected as appropriate as long as a particle size differentiation means, such as FFF, can separate particles belonging to one particle group with a certain average particle size from those belonging to another particle group with another average particle size.

With the above-described configuration, it is possible to form label complexes each of which includes particle 1 via first binding portion 12 and second binding portion 22. In addition, it is also possible to differentiate among the types of the label complexes, based on the particle size mainly attributable to particle body 11 as well as the label property of labeling portion 21. As a result, it is possible to distinguish among the types of biomarkers 3 respectively corresponding to the label complexes.

### (3-3. Particle Groups and Label Groups)

In the measurement method according to the present embodiment, the label complexes are separated based on the particle size and then distinguished based on the label property. Next, sets (groups) of particles 1 defined by the particle size and the label property will be described.

FIG. 3 is a view for explaining particle groups and label groups. In FIG. 3, groups including particles 1a to 1d and labels 2a to 2d capable of specifically binding to four types of biomarkers 3a to 3d, respectively, are illustrated.

In the present embodiment, a “particle group” is a set of particles 1 that bind to labels 2 with a particular label property. In FIG. 3, a first particle group and a second particle group are illustrated.

Similarly, a “label group” is a set of labels 2 corresponding to a certain particle group. In FIG. 3, a first label group corresponding to the first particle group and a second label group corresponding to the second particle group are illustrated. Particle 1 of the first particle group and label 2 of the first label group bind to each other via biomarker 3. Particle 1 of the second particle group and label 2 of the second label group bind to each other via biomarker 3.

Label 2 (2a, 2b) of the first label group and label 2 (2c, 2d) of the second label group are different from each other in label property. Referring to FIG. 3, label 2 of the first label group includes a labeling portion 21a having a certain label property. Label 2 of the second label group includes a labeling portion 21c having a label property that is different from that of the first label group.

Each of the first particle group and the second particle group (hereinafter also referred to as “each particle group”) includes a plurality of particle subgroups.

Each particle subgroup includes particles 1 that are the same in particle size and labeled with labels 2 having the same label property. Referring to FIG. 3, for example, a particle subgroup a consists of particles 1a that are the same in particle size and labeled with labels 2a. Each label group includes a plurality of label subgroups respectively corresponding to a plurality of particle subgroups of each particle group. A set of labels 2a corresponding to particle subgroup a is referred to as a “label subgroup” a. Particle subgroup a and label subgroup a are collectively referred to as a “particle-label subgroup” a. The number of types of particle-label subgroups corresponds to the number of types of biomarkers 3 that are distinguishable by them.

Particles 1 of different particle subgroups bind to different types of biomarkers 3, and, therefore, they have different first binding portions 12. Different label subgroups bind to different types of biomarkers 3, and, therefore, they have different second binding portions 22. In other words, particles 1 of different particle subgroups have respective first binding portions 12 that are structurally capable of specifically binding to different types of biomarkers 3. Similarly, labels 2 of different label subgroups have respective second binding portions 22 structurally capable of specifically binding to biomarkers 3 to which particles 1 of the corresponding particle subgroups in the particle group specifically bind. Referring to FIG. 3, particles 1a to 1d include first binding portions 12a to 12d, respectively. Referring to FIG. 3, labels 2a to 2d include second binding portions 22a to 22d, respectively.

In each particle group, the plurality of particle subgroups are different from each other in particle size. Referring to FIG. 3, in the first particle group, the diameter of a particle body 11a of particle 1a in particle subgroup a is larger than the diameter of a particle body 11b of particle 1b in a particle subgroup b. In the second particle group, the diameter of particle body 11a of particle 1c in a particle subgroup c is larger than the diameter of particle body 11b of particle 1d in a particle subgroup d. As a result, the particle subgroups within each particle group can be differentiated from each other based on the particle size.

Next, the second particle group and the second label group will be described more. A particle-label subgroup c corresponding to the second particle group and particle-label subgroup a have a common particle size, but they are different from each other in the label property of labeling portion 21. Referring to FIG. 3, particle-label subgroup c and particle-label subgroup a can be differentiated from each other based on the label property. Similarly, a particle-label subgroup d corresponding to the second particle group and a particle-label subgroup b have a common particle size, but they are different from each other in the label property of labeling portion 21. As a result, particle-label subgroup d and particle-label subgroup b can be differentiated from each other based on the label property.

In this way, a label complex corresponding to a particle-label subgroup corresponding to a certain type of biomarker 3 can be distinguished from a label complex corresponding to another particle-label subgroup, based on the particle size and the label property. More specifically, when the above-mentioned certain particle-label subgroup and the above-mentioned another particle-label subgroup correspond to the same particle group, they can be separated from each other based on the particle size. When the above-mentioned certain particle-label subgroup and the above-mentioned another particle-label subgroup correspond to different particle groups, they can be distinguished from each other based on the label property. As a result, the types of biomarkers 3 can be differentiated from each other based on the particle size and the label property.

Alternatively, as a matter of course, the number of particle subgroups in each particle group and the number of label groups may be 3 or more, respectively, and similarly, the number of particle groups and the number of label subgroups in each label group may also be 3 or more, respectively. For example, when the number of particle subgroups in each particle group is from 10 to 20 and the number of label groups is from 4 to 5, it is possible to simultaneously measure 40 to 100 types of biomarkers. When the number of particle subgroups in each particle group is from 10 to 20 and the number of label groups is about 20, it is possible to simultaneously measure about 200 to 400 types of biomarkers. In this way, with the combination of the variation in label property and the variation in particle size, the number of types of biomarkers that can be simultaneously measured can be increased.

In the example illustrated in FIG. 3, two patterns of particle sizes and two patterns of label properties are combined in a matrix to form four patterns of particle-label subgroups, but as a matter of course, the particle size and the label property are not necessarily combined in a matrix. For example, the particle size patterns for the particle subgroups in the first particle group may be different from the particle size combinations for the subgroups in the second particle group. However, when the particle size patterns and the label property patterns are combined in a matrix to form particle-label subgroups, it is possible to minimize the number of particle size patterns and the number of label property patterns necessary for forming all the particle-label subgroups, which is an advantage. As a result, it is possible to make the utmost use of the capability of separation unit 8 of separating among the particle sizes as well as the capability of detection unit 9 of separating among the detected values of the label properties, and thereby form the maximum number of particle-label subgroups. In other words, it is possible to maximize the number of types of biomarkers that can be distinguished from each other.

### (3-4. Method for Binding Particle, Biomarker, and Label Together)

Next, the method for binding particle 1, biomarker 3, and label 2 together at pretreatment unit 71 will be described. In an example, the method is executed manually by a user with the use of a typical molecular biology experiment instrument, but it may also be executed by an automated pretreatment apparatus. In other words, pretreatment unit 71 may be an automated pretreatment apparatus, or may be an experiment instrument operated by a user.

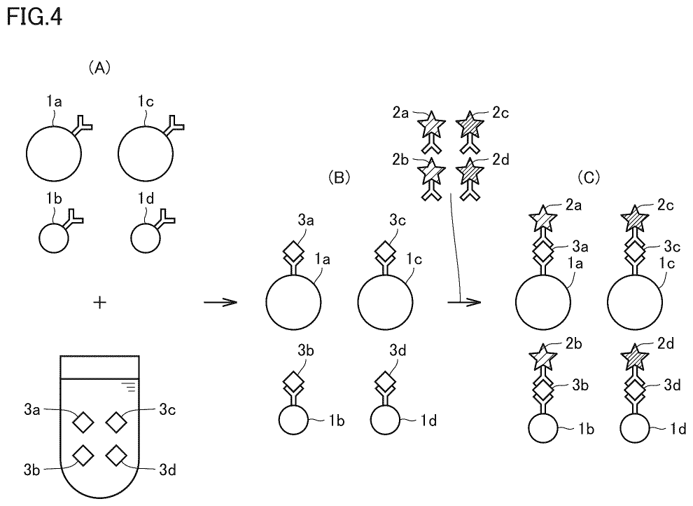

FIG. 4 is a view for explaining a method for binding a particle, a biomarker, and a label together.

Firstly, a user prepares a specimen that contains a biomolecule, which is referred to as a biomarker (measurement target). The specimen is a specimen derived from a living body, such as urine and/or blood of a subject, for example. The specimen may be prepared and/or purified in advance as appropriate. In an example, the specimen is contained in a vessel that is usually used in preparation of biological specimens, such as a microtube.

Then, the user mixes particles 1 to the specimen. For example, to the vessel containing the specimen, the user adds and mixes a solution including particles 1, a certain number of them for each particle subgroup, to prepare a mixed solution. Each particle 1 is made in such a manner that it can bind to biomarker 3 (measurement target). As a result, biomarkers 3 (measurement target) contained in the specimen can bind to corresponding particles 1. In an example, the number of particles 1 mixed here is sufficiently greater than the number of biomarkers 3 so that substantially all the biomarkers 3 (measurement target) can bind to particles 1. As a result, in the mixed solution, each biomarker 3 (measurement target) is present in the form of a biomarker complex.

Then, the user further mixes labels 2 to the mixed solution. As a result, labels 2 specifically bind to biomarkers 3 each in the form of a biomarker complex. In an example, the number of labels 2 mixed here is sufficiently greater than the number of biomarkers 3 so that substantially all the biomarkers 3 (measurement target) can be labeled. As a result, in the mixed solution, each biomarker 3 (measurement target) is present in the form of a label complex.

The above-described labeling method that involves sandwiching a measurement target substance (here, it is biomarker 3) with substances capable of specifically binding to the substance (here, they are first binding portion 12 of particle 1 and second binding portion 22 of label 2) is generally called a sandwich method. With the sandwich method, it is possible to specifically label multiple types of biomarkers 3 simultaneously in one step, only by adding multiple types of labels 2 corresponding to the multiple types of biomarkers 3.

By injecting the mixed solution prepared in the above-described manner into measurement apparatus 5, it is possible to perform separation at separation unit 8 based on the particle size of the label complexes and detect the label property at detection unit 9.

The mixed solution also includes free-form biomarkers which are not measurement targets, but they are smaller than the label complex with the smallest particle size, so they are separated at separation unit 8 from the label complex (measurement target). Because of this, they do not affect the results of label complex detection. The mixed solution also includes particles 1 to each of which biomarker 3 (measurement target) and label 2 are not bound, but because they are not labeled, they are not detected at detection unit 9. Because of this, they do not affect the results of label complex detection.

Next, actual results of separation based on the particle size and results of label property detection at measurement apparatus 5 will be described.

### (3-5. Results of Separation Based on Particle Size and Results of Label Property Detection)

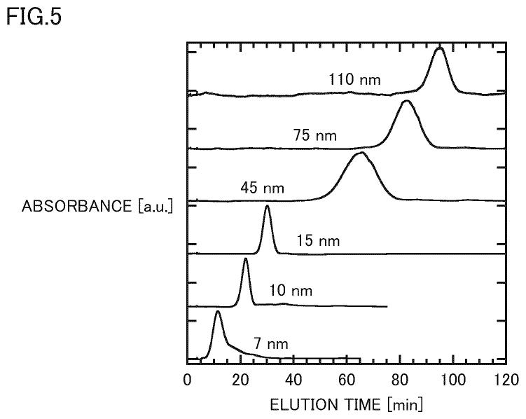

FIG. 5 is a view showing results of separation based on the particle size. In FIG. 5, the horizontal axis represents elution time. The elution time is the time from injection of the mixed solution into specimen injection unit 72 to detection of a certain component. The vertical axis represents absorbance after baseline correction. Referring to FIG. 5, particles 1 with a diameter of 7 nm, 10 nm, 15 nm, 45 nm, 75 nm, and 110 nm, respectively, were successfully detected with substantially no overlap in elution time. In other words, it is possible to separate label complexes based on the particle size.

FIG. 6 is a view showing results of label property detection. In FIG. 6, the horizontal axis represents wavelength and the vertical axis represents fluorescence intensity. Referring to FIG. 6, three fluorescence spectra were successfully detected in such a manner that they can be differentiated from each other. In other words, it is possible to separate label complexes based on the fluorescence spectra, which are the label properties.

From the results shown in FIG. 5 and FIG. 6, it is possible to differentiate among the particle-label subgroups based on the particle size and the label property. As a result, it is possible to distinguish the type of the detected biomarker.

### (3-6. Measurement Process)

Next, the process for executing the measurement method according to the embodiment will be described.

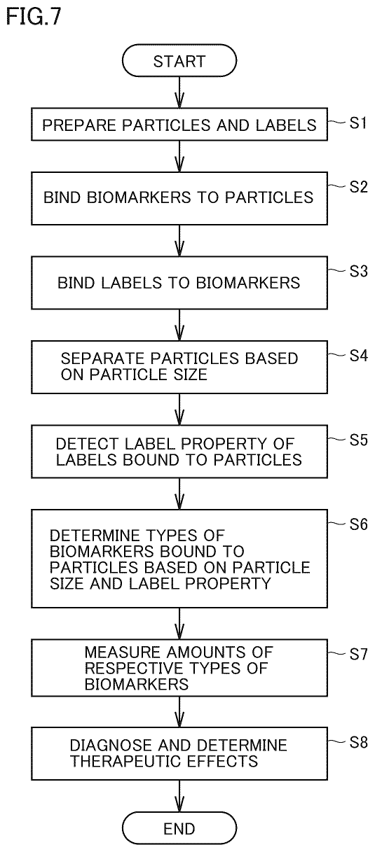

FIG. 7 is a flowchart illustrating a measurement process according to the present embodiment. The steps illustrated in FIG. 7 are executed with the use of measurement system 100.

In the drawing, “S” is an abbreviation of “STEP”.

In S1, the user prepares particles 1 belonging to a first particle group and particles 1 belonging to a second particle group. The user also prepares labels 2 belonging to a first label group corresponding to the first particle group, and labels 2 belonging to a second label group corresponding to the second particle group.

In S2, with the use of pretreatment unit 71, the user mixes a specimen, particles 1 of the first particle group, and particles 1 of the second particle group to allow biomarkers 3 to specifically bind to particles 1 of the first particle group and particles 1 of the second particle group.

In S3, with the use of pretreatment unit 71, the user mixes the mixed solution prepared in S2, labels 2 of the first label group, and labels 2 of the second label group to allow labels 2 of the first label group and labels 2 of the second label group to specifically bind to biomarkers 3.

In S4, processor 40 separates among particles 1 of the first particle group and also among particles 1 of the second particle group, based on the particle size. In an example, processor 40 introduces the mixed solution that was injected from injection unit 72, into separation unit 8, and at separation unit 8, separates between label complexes and lone particles 1 contained in the mixed solution based on particle size. For example, when separation unit 8 adopts centrifugal FFF, molecules with small particle sizes flow out first. Accordingly, after free-form labels 2 in the mixed solution flow out, fractionation takes place based on the particle size in such a manner that lone particles 1 or label complexes including particles 1 with the smallest particle size flow out, then lone particles 1 or label complexes including particles 1 with the second smallest particle size flow out, then lone particles 1 or label complexes including particles 1 with the third smallest particle size flow out, and on and on.

It should be noted that the processes S2 and S3 may be executed in such a manner that processor 40 controls an apparatus (for example, an automated pretreatment apparatus) capable of executing an equivalent process to the above-mentioned operation executed by the user. Moreover, S2 and S3 may be executed in the reverse order, or may be executed simultaneously. Specifically, it is also possible to mix the specimen with labels 2 in advance to allow labels 2 and biomarkers 3 to bind to each other, and then mix particles 1 thereto to allow particles 1 to bind to biomarkers 3. Alternatively, it is also possible to simultaneously mix the specimen, particles 1, and labels 2 together to allow both particles 1 and labels 2 to bind to biomarkers 3 in one step.

In S5, processor 40 detects the label property of label 2 bound via biomarker 3 to each of particles 1 of the first particle group and particles 1 of the second particle group thus separated based on the particle size. For example, when detection unit 9 is a plurality of fluorescence detectors or multi-wavelength fluorescence detectors, processor 40 irradiates particles 1 with excitation light and detects the intensity of fluorescence thus emitted.

In S6, based on the particle size and the label property, processor 40 determines the type of biomarker 3 bound to each of particles 1 of the first particle group and particles 1 of the second particle group.

In S7, processor 40 measures the amounts of respective types of biomarkers 3 based on the results of determination of the types of biomarkers 3. The amount of respective types of biomarkers 3 is, for example, the concentrations or the number of respective biomarkers 3 in the specimen.

In an example of S6 and S7, processor 40 determines the types of biomarkers 3 corresponding to labels 2 that emitted fluorescence, based on the fluorescence intensity, and measures the amounts of respective types of biomarkers 3.

More specifically, in the case to detect fluorescence for one particle at one time (namely, for one lone particle 1 or one label complex at one time), processor 40 distinguishes a label 2 corresponding to the fluorescence intensity, and distinguishes the type of biomarker 3 corresponding to the label 2. Then, every time it distinguishes the type of a new biomarker 3, it adds the number of biomarker 3 thus distinguished. As a result, “the number of biomarkers 3 included in the label complexes in the mixed solution” is determined. In an example, the number of particles 1 and the number of labels 2 are adjusted to be enough for allowing all the biomarkers 3 in the mixed solution to form label complexes. Accordingly, it is conceivable that “the number of biomarkers 3 included in the label complexes in the mixed solution” corresponds to “the number of biomarkers 3 contained in the specimen”. Based on “the number of biomarkers 3 contained in the specimen” and “the entire amount of the specimen before preparation of the mixed solution”, processor 40 can determine “the concentration of biomarkers 3 contained in the specimen”.

On the other hand, when detection unit 9 simultaneously detects fluorescence of a plurality of particles, processor 40 calculates the number of labels 2 having respective label properties (fluorescence intensity at a certain wavelength, for example), from an assemblage of fluorescence spectra of the plurality of particles. As a more specific example, processor 40 distinguishes the fluorescence intensity or the peak area for the respective fluorescence spectra of the plurality of particles at a certain wavelength, and distinguishes the types of biomarkers 3 corresponding to labels 2, as well as the number of them.

Even in the case where detection unit 9 detects fluorescence for one particle at one time, it may output an assemblage of fluorescence spectra of the plurality of particles, namely, all the detection results, within a certain range, added up. In this case, processor 40 executes processing in a similar manner to when simultaneously detecting fluorescence for the plurality of particles. The above-mentioned certain range is a range within which the particle sizes are equivalent to each other, for example. The above-mentioned range within which the particle sizes are equivalent to each other is, for example, defined in advance based on the amount of time elapsed from the start of the separation process at separation unit 8.

In S8, the user makes a disease diagnosis and/or a therapeutic effect determination based on the amounts of respective types of biomarkers 3. For example, based on the combination of the amounts of multiple types of biomarkers 3 potentially involved with a certain disease and treatment efficacy, determination is made on the presence or absence of the disease, the severity of the disease, the rate of progress of the disease, the efficacy of treatment, and/or the like. It should be noted that the combination of the amounts of multiple types of biomarkers 3 may be stored in memory 41 so that processor 40 can automatically execute processing equivalent to diagnosis performed by the user.

In this way, with the measurement method according to the present embodiment, it is possible to differentiate and detect many types of biomarkers 3 by using a combination of particles 1 having different particle sizes and labels 2 having different label properties. As a result, it is possible to simultaneously measure many types of biomarkers 3. It is also made possible to make a disease diagnosis or a therapeutic effect determination based on the amounts of respective types of biomarkers that were measured simultaneously. That is, it is made possible to make a diagnosis based on multiple types of biomarkers, in an easy and simple manner.

The above description explains a configuration for distinguishing among the types of biomarkers 3 based on the particle size and the label property, but, as a matter of course, it is also possible to distinguish among biomarkers 3 based solely on the particle size. For example, it is possible to distinguish among the types of biomarkers 3 corresponding to respective subgroups in the first particle group, by solely using the first particle group and the first label group illustrated in FIG. 3. In this case, it is possible to distinguish among the types of biomarkers 3 corresponding to the number of different particle sizes. In this way, a technique for simultaneously measuring multiple types of biomolecules based on the difference in particle size can be provided.

### 4. Measurement Method According to Variation 1

The detected value (the peak area, for example) of the label property (fluorescence intensity, for example) detected in S5 of FIG. 7 is mainly attributed to labeling portion 21. However, there is a possibility that a component attributed to other parts of the label complex can be included. Such a component of a detected value attributed to a part other than a measurement target is generally referred to as “background”. In the measurement method according to the present embodiment, it may be preferable to correct such influence, imposed on a detected value, by the background, especially those imposed by particle body 11 which makes up a large part of a label complex. In Variation 1 of the present embodiment, a configuration for canceling the background to correct the detected value will be described.

### (4-1. Measurement Process Including Correction Process)

Next, a measurement method according to Variation 1 of the present embodiment will be described. The measurement method according to Variation 1 includes a process to correct the detected value that was obtained by the label property detection process. In Variation 1, for differentiating the detected value before correction from the detected value after correction, they are referred to as a pre-correction detected value and a post-correction detected value, respectively.

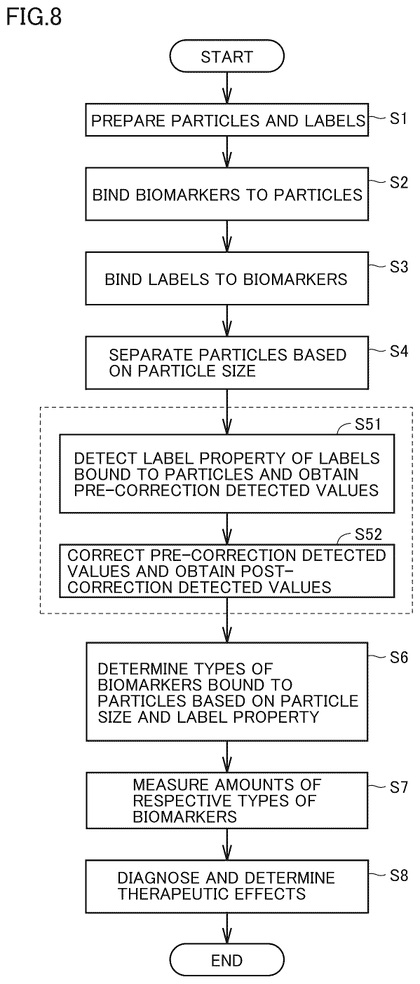

FIG. 8 is a flowchart illustrating a measurement process according to Variation 1. The steps illustrated in FIG. 8 are executed with the use of measurement system 100. In FIGS. 8, S51 and S52 are executed instead of S5 in FIGS. 7. S1 to S4 and S6 to S8 in FIG. 8 correspond to S1 to S4 and S6 to S8 in FIG. 7, respectively. The description of steps in FIG. 8 that overlap with FIG. 7 is not repeated.

In S51 of FIG. 8, processor 40 detects the label property of label 2 bound to particle 1 and obtains a pre-correction detected value.

In S52, processor 40 corrects the pre-correction detected value and obtains a post-correction detected value.

Next, as an specific aspect of correction in S52, a correction method using a first correction particle and a correction method using a second correction particle will be described.

### (4-2. Correction Method Using First Correction Particle)

In a first step of this correction, firstly, first correction particles having sizes corresponding to the particle sizes of a plurality of particle subgroups in each particle group are prepared. To each first correction particle, labeling portion 21 does not bind.

In an example, the first correction particle is a particle that affects the detected value in an equivalent manner to particle 1, and typically, it is an object equivalent to particle 1. An object equivalent to particle 1 is, for example, an object with the same size, composition, and/or structure. The first correction particle includes particle body 11, which is a part that is considered to have relatively great influence on the detected value of the label property, second to labeling portion 21. The first correction particle may further include first binding portion 12 that is considered to have relatively small influence on the detected value of the label property. With this, it is possible to perform more accurate correction.

In an example, the label property is fluorescence intensity, and in this case, light scattered at the surface of particle body 11 can affect the detected value of the label property. More specifically, influence of scattered light manifests itself as a peak area attributed to the scattered light.

In the next step, the label property of the first correction particle “not bound with labeling portion 21” is detected. To the first correction particle in this state, biomarker 3 and second binding portion 22 each of which is a part that is considered to have relatively small influence on the detected value of the label property, may be bound. With this, it is possible to perform more accurate correction, taking into consideration the influence of biomarker 3 and second binding portion 22.

In an example, the first correction particle is measured in the same manner as in the measurement of mixed specimens including the label complex (measurement target). Specifically, the first correction particles are introduced from injection unit 72 and separated at separation unit 8 based on the size, and then the label properties are detected at detection unit 9.

In the last step, for each particle size, the pre-correction detected value of the label property of labeling portion 21 bound to each of particle 1 of the first particle group and particle 1 of the second particle group is corrected based on the detected value of the label property of the first correction particle, and, thereby, a post-correction detected value is obtained.

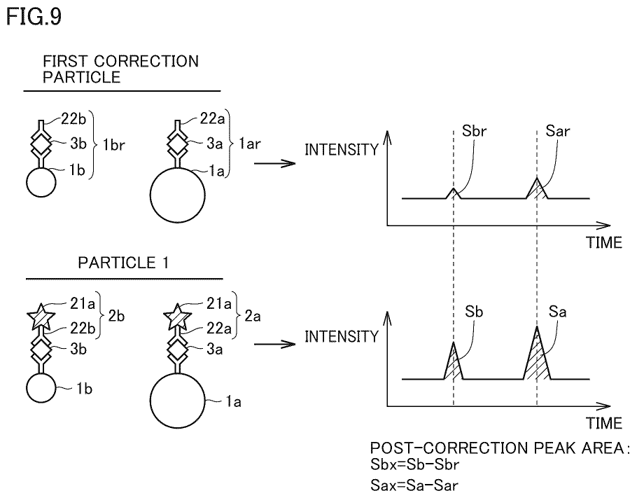

FIG. 9 is a view for explaining a specific example of correction with the use of a first correction particle. In the example illustrated in FIG. 9, the label property is fluorescence intensity. The graphs in FIG. 9 show the detection intensity at a certain fluorescence wavelength along with the elution time. The horizontal axis in each graph in FIG. 9 represents the amount of time elapsed from injection at injection unit 72. That is, the horizontal axis is correlated with the particle size. The vertical axis represents fluorescence intensity at a certain fluorescence wavelength 2a. More specifically, it is the fluorescence intensity at a characteristic fluorescence wavelength for the fluorescence spectrum of labeling portion 21a for particle 1a, 1b (measurement target). The characteristic fluorescence wavelength for the fluorescence spectrum of labeling portion 21a is, for example, a fluorescence wavelength that corresponds to a peak of the fluorescence spectrum for labeling portion 21a and at which substantially no fluorescence spectra for labeling portions 21 of other particles are detected.

In FIG. 9, a peak with a peak area Sar is detected for a first correction particle 1ar not bound with labeling portion 21, and a peak with a peak area Sbr is detected for a first correction particle 1br not bound with labeling portion 21. The first correction particle 1ar is an object equivalent to particle 1a. The first correction particle 1br is an object equivalent to particle 1b. Peak area Sar, Sbr corresponds to background.

Also, results of label property detection for a label complex including labeling portion 21a and particle 1a and for a label complex including labeling portion 21a and particle 1b are provided as a peak with a peak area Sa and a peak with a peak area Sb, respectively. Peak area Sa and peak area Sb correspond to examples of the “pre-correction detected value”.

From the pre-correction detected value, the detected value of first correction particle 1ar or the detected value of first correction particle 1br is subtracted, to perform correction. Accordingly, a post-correction peak area Sax for the label complex including particle 1a is calculated as Sax=Sa−Sar. Also, a post-correction peak area Sbx for the label complex including particle 1b is calculated as Sbx=Sb−Sbr. Peak area Sax and peak area Sbx correspond to examples of the “post-correction detected value”.

That is, in the example illustrated in FIG. 9, the influence of light scattered at the surface of particle body 11 can be canceled. By this, as the post-correction peak area, a component correlated with the number of detected labeling portions 21a remains. As a result, with the post-correction peak area, the accuracy of counting the number of detected labeling portions 21a is enhanced.

With the background data in the pre-correction detected value being thus eliminated with the use of the first correction particle, the post-correction detected value becomes mainly attributed to the label property of labeling portion 21. Thereby, based on the post-correction detected value, the number of respective types of biomarkers 3 is determined more accurately. As a result, accuracy of quantification of biomarkers 3 in the measurement method according to the present embodiment is enhanced.

It should be noted that the first correction particle used here has a particle size equivalent to that of particle 1 (measurement target), so the range of variations in the size of the particles (measurement target) does not become smaller. As a result, it is possible to correct background while maintaining the number of types of biomarkers distinguishable from each other as high as possible.

### (4-3. Other Configurations of Correction Method)

In the above-described correction using the first correction particle, it is necessary to separately perform measurement of the first correction particle and measurement of the label complex (measurement target). Next, a method to perform correction in a single measurement by adding a correction particle as an internal standard to the label complex (measurement target) will be described.

In a first step of this correction, firstly, second correction particles having particle sizes different from the particle sizes of respective particle subgroups in each particle group are prepared.

To each second correction particle, labeling portion 21 does not bind. In an example, the second correction particle includes particle body 11 the size of which is different from the size of particle bodies 11 of the plurality of particle subgroups in each particle group.

In the next step, the label property of the second correction particle not bound with labeling portion 21 is detected. In an example, the second correction particle is mixed with the mixed specimen containing the label complex including particle 1 (measurement target), and then introduced from injection unit 72. Subsequently, the second correction particle and the label complex (measurement target) are classified by separation unit 8, and then at detection unit 9, the respective label properties are detected. By this, the detected value of the second correction particle and the pre-correction detected value of the label complex (measurement target) can be simultaneously obtained.

In the last step, for each of particle 1 of the first particle group and particle 1 of the second particle group, the pre-correction detected value of the label property of labeling portion 21 thus bound is corrected based on the detected value of the label property of the second correction particle, and, thereby a post-correction detected value is obtained.

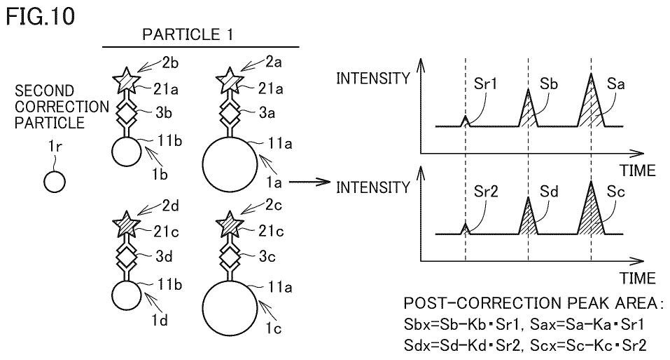

FIG. 10 is a view for explaining a specific example of correction with the use of a second correction particle. In the example illustrated in FIG. 10, the label property is fluorescence intensity. The horizontal axis in each graph in FIG. 10 represents the amount of time elapsed from injection at injection unit 72. Accordingly, the horizontal axis is correlated with the particle size. The vertical axis represents fluorescence intensity at a certain wavelength. More specifically, the vertical axis in the upper graph in FIG. 10 represents the fluorescence intensity at a characteristic fluorescence wavelength λa for the fluorescence spectrum of labeling portion 21a for particle 1a, 1b. The vertical axis in the lower graph in FIG. 10 represents the fluorescence intensity at a characteristic fluorescence wavelength λc for the fluorescence spectrum of labeling portion 21c of particle 1c, 1d.

FIG. 10 gives results of measurement performed after simultaneous injection of label complexes including particles 1a to 1d (measurement target) and second correction particles 1r into measurement apparatus 5.

Specifically, the upper graph in FIG. 10, which is a “graph of fluorescence intensity at wavelength λa”, shows a peak with a peak area Sr1 detected for second correction particle 1r. It also shows results of label property detection for a label complex including labeling portion 21a and particle 1a as well as a label complex including labeling portion 21a and particle 1b, as a peak with peak area Sa and a peak with peak area Sb, respectively.

The lower graph in FIG. 10, which is a “graph of fluorescence intensity at wavelength λc”, shows a peak with a peak area Sr2 detected for second correction particle 1r. It also shows results of label property detection for a label complex including labeling portion 21c and particle 1c as well as a label complex including labeling portion 21c and particle 1d, as a peak with a peak area Sc and a peak with a peak area Sd, respectively. Peak areas Sa to Sd correspond to examples of the “pre-correction detected value”.

The pre-correction detected value is corrected with the use of a correction coefficient Ka to Kd calculated based on peak area Sr1, Sr2 for second correction particle 1r. Correction coefficient Ka, Kb is a numerical value that indicates the extent of change of the peak area for particle body 11a, 11b with respect to peak area Sr1 for the second correction particle. Correction coefficient Kc, Kd is a numerical value that indicates the extent of change of the peak area for particle body 11a, 11b with respect to peak area Sr2 for the second correction particle. Correction coefficient Ka to Kd is calculated with the Mie scattering model or the Rayleigh scattering model. As correction coefficient Ka to Kd, a value that is determined based on detected values resulting from measurement performed in advance on unlabeled particles 1 may be used. Thereby, by using the peak area for second correction particle 1r, it is possible to calculate the influence imposed on the peak area by a measurement-target particle that has a different particle size from second correction particle 1r.

With the use of the correction coefficient thus calculated, post-correction peak area Sax for a label complex including particle 1a is calculated as Sax=Sa−Ka×Sr1. Post-correction peak area Sbx for a label complex including particle 1b is calculated as Sbx=Sb−Kb×Sr1. A post-correction peak area Scx for a label complex including particle 1c is calculated as Scx=Sc−Kc×Sr2. A post-correction peak area Sdx for a label complex including particle 1d is calculated as Sdx=Sdp31 Kd×Sr2.

In the example illustrated in FIG. 10, the influence of light scattered at the surface of particle body 11 of a certain size (background) is calculated based on the peak area for the second correction particle (internal standard). By canceling the background, it is possible to make the post-correction detected value mainly attributed to the label property of the labeling portion. As a result, with no need for additional cost or time for separately measuring the background, it is possible to enhance the accuracy of quantification of biomarkers 3. Thus, with the use of the second correction particle, it is possible to enhance the accuracy of quantification of biomarkers 3, in an easy and simple manner.

The above-described correction is also applicable when the peaks in FIG. 9 and FIG. 10 are the detection results for a combined total of label properties of a plurality of particles, as well as when they are the detection results for the label property of one particle.

### 5. Variation 2

As described above, with the measurement method according to the present embodiment, it is possible to distinguish among biomarkers 3 by differentiating among label complexes based on the particle size and the label property of label 2. Meanwhile, by configuring the particles to have their own properties (particle property), it is also possible to distinguish among biomarkers 3 by differentiating among label complexes based on the particle size and the particle property.

### (5-1. Structure of Particles and Labels According to Variation 2)

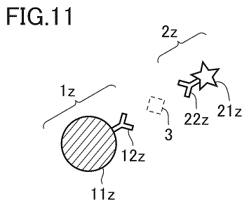

FIG. 11 is a view for explaining the structure of a particle and a label according to Variation 2. The particle and the label according to Variation 2 are characteristically different from particle 1 and label 2 according to both the embodiment and Variation 1, so, in Variation 2, they are referred to as a particle 1z and a label 2z, respectively. Description regarding FIG. 11 will only mention parts that are different from FIG. 2, which describes the structure of the particle and the label according to the embodiment.

Particle 1z includes a particle body 11z, as well as a first binding portion 12z that is capable of specifically binding to biomarker 3.

Particle body 11z has a particle property.

The particle property is not particularly limited as long as it is a property that allows for differentiating among different types of particles 1z corresponding to different types of biomarkers 3. In an example, the particle property is a fluorescence spectrum pattern. The fluorescence spectrum pattern is a pattern of fluorescence intensity with respect to fluorescence wavelengths, and more specifically, it is the shape of a graph of fluorescence intensity with respect to fluorescence wavelengths. When there is a commonality between fluorescence spectrum patterns (particle property), it means that there is a commonality between patterns of fluorescence intensity with respect to wavelengths, but the fluorescence intensity at a particular wavelength is not necessarily the same as each other. When the particle property is a fluorescence spectrum pattern, the substance forming the outer surface of particle body 11z is preferably a substance that can be stained (silica, for example). The particle property may be at least one of a radiation spectrum pattern and an absorption spectrum pattern. In the measurement method according to Variation 2, the types of biomarkers 3 are distinguished from each other based on the difference in the particle property.

First binding portion 12z is bound to particle body 11z. First binding portion 12z is a binding site on particle 1z for specifically binding to a certain type of biomarker 3.

Label 2z includes a labeling portion 21z, as well as a second binding portion 22z that is capable of specifically binding to biomarker 3.

Labeling portion 21z is a portion that includes a label substance for labeling particle 1z to which biomarker 3 is bound. The label substance exhibits a certain label property. As described below, labeling portion 21z of label 2z is simply required to indicate binding of particle 1z to biomarker 3, and it is not required to indicate the type of biomarker 3. Accordingly, regardless of the types of biomarkers 3 to which labels 2z bind, labeling portions 21z of them may be equivalent to each other.

Second binding portion 22z is bound to labeling portion 21z. Second binding portion 22z is a binding site on label 2z for specifically binding to a certain type of biomarker 3.

### (5-2. Particle Groups and Label Groups According to Variation 2)

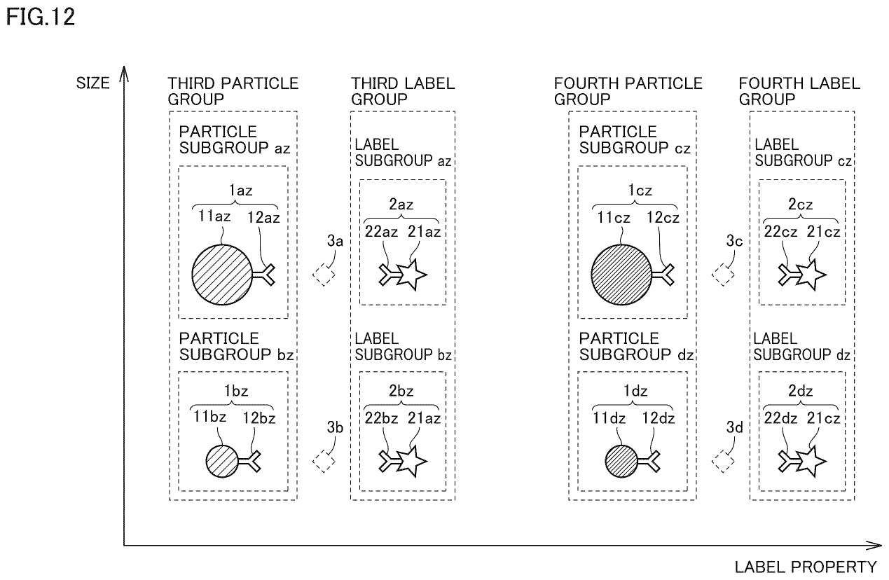

FIG. 12 is a view for explaining particle groups and label groups according to Variation 2. In FIG. 12, sets of particles 1az to 1dz and labels 2az to 2dz capable of specifically binding to four types of biomarkers 3a to 3d, respectively, are illustrated. Description regarding FIG. 12 will only mention parts that are different from FIG. 3, which describes the particle groups and the label groups according to the embodiment.

In Variation 2, a “particle group” is a set of particles 1z with a particular particle property. In FIG. 12, a third particle group and a fourth particle group are illustrated. Similarly, a “label group” is a set of labels 2z corresponding to a certain particle group. In FIG. 12, a third label group corresponding to the third particle group and a fourth label group corresponding to the fourth particle group are illustrated. Particle 1z of the third particle group and label 2z of the third label group bind to each other via biomarker 3. Particle 1z of the fourth particle group and label 2z of the fourth label group bind to each other via biomarker 3.

Particle 1z of the third particle group and particle 1z of the fourth particle group are different from each other in particle property. Referring to FIG. 12, particles 1az, 1bz of the third particle group include particle bodies 11az, 11bz that have a common particle property. Particles 1cz, 1dz of the fourth particle group include particle bodies 11cz, 11dz that have a common particle property.

Each of the third particle group and the fourth particle group (hereinafter also referred to as “each particle group”) includes a plurality of particle subgroups.

Each particle subgroup includes particles 1z that have a common particle size and a common particle property. Referring to FIG. 12, for example, a particle subgroup az consists of particles 1az that are the same in particle size and particle property. A set of labels 2az corresponding to particle subgroup az is referred to as a “label subgroup” az. Particle subgroup az and label subgroup az are collectively referred to as a “particle-label subgroup” az. The number of particle-label subgroups corresponds to the number of types of biomarkers 3 that are distinguishable by them.

Particles 1z of different particle subgroups bind to different types of biomarkers 3, and, therefore, they have different first binding portions 12z. Different label subgroups bind to different types of biomarkers 3, and, therefore, they have different second binding portions 22z. In other words, particles 1z of different particle subgroups have respective first binding portions 12z that bind to different types of biomarkers 3. Similarly, labels 2z of different label subgroups have respective second binding portions 22z that bind to different types of biomarkers 3. Referring to FIG. 3, particles 1az to 1dz include first binding portions 12az to 12dz, respectively. Referring to FIG. 3, labels 2az to 2dz include second binding portions 22az to 22dz, respectively. It should be noted that first binding portion 12z and second binding portion 22z according to Variation 2 may be equivalent to first binding portion 12 and second binding portion 22 according to the embodiment, respectively, when they bind to the same type of biomarker. Specifically, first binding portions 12az to 12dz may be equivalent substances to first binding portions 12a to 12d of particles 1 according to the embodiment, respectively (for example, substances having the same molecular structure). Second binding portions 22az to 22dz may be equivalent substances to second binding portions 22a to 22d of labels 2 according to the embodiment, respectively (for example, substances having the same molecular structure).

In each particle group, the plurality of particle subgroups are different from each other in particle size. Referring to FIG. 3, in the third particle group, the diameter of particle body 11az of particle 1az in particle subgroup az is larger than the diameter of particle body 11bz of particle 1bz in a particle subgroup bz. In the fourth particle group, the diameter of particle body 11cz of particle 1cz in a particle subgroup cz is larger than the diameter of particle body 11dz of particle 1dz in a particle subgroup dz. As a result, the particle subgroups within each particle group can be differentiated from each other based on the particle size. It should be noted that in an example, the diameter of particle body 11az is equivalent to the diameter of particle body 11cz. Similarly, the diameter of particle body 11bz is equivalent to the diameter of particle body 11dz.

Next, the fourth particle group and the fourth label group will be described more. A particle-label subgroup cz corresponding to the fourth particle group and particle-label subgroup az have a common particle size, but they are different from each other in particle property. As a result, particle-label subgroup cz and particle-label subgroup az can be differentiated from each other based on the particle property. Similarly, a particle-label subgroup dz corresponding to the fourth particle group and a particle-label subgroup bz have a common particle size, but they are different from each other in particle property. As a result, particle-label subgroup dz and particle-label subgroup bz can be differentiated from each other based on the particle property.

In this way, particles 1z included in a certain particle subgroup can be distinguished from particles 1z included in another particle subgroup based on the particle size and the particle property. More specifically, when the above-mentioned certain particle subgroup and the above-mentioned another particle subgroup belong to the same particle group, they can be separated from each other based on the particle size. When the above-mentioned certain particle subgroup and the above-mentioned another particle subgroup belong to different particle groups, they can be distinguished from each other based on the particle property.

As described above, in Variation 2, it is possible to distinguish particles 1z

included in a certain particle subgroup from particles 1z in another particle subgroup based on the particle size and the particle property. As a result, based on the particle size and the particle property, it is possible to distinguish among the types of biomarkers 3 corresponding thereto. Therefore, it is not necessary to rely on the label property of label 2z when distinguishing among different types of biomarkers 3 corresponding thereto. Specifically, label 2z is simply required to indicate binding of biomarker 3 with particle 1z regardless of the type of biomarker 3.

Accordingly, in Variation 2, the label properties of labels 2z may be the same between label subgroups. More specifically, the detected value of the label property of the third label group may be equivalent to the detected value of the label property of the fourth label group. In the example illustrated in FIG. 9, a labeling portion 21az may be an object that exhibits the same detected value as a labeling portion 21cz, and more specifically, the former may be the same object as the latter. When the label properties of all the labeling portions are equivalent to each other in this way, preparation of the labeling portions can become less complicated.

However, it should be noted that for each of the particles of the third particle group and the particles of the fourth particle group, the detected value of the particle property needs to be distinguishable from the detected value of the label property. The particle property and the label property may be configured to be properties of the same type that are distinguishable from each other. For example, the particle property and the label property may be fluorescence spectra that are distinguishable from each other. Alternatively, the particle property and the label property may be different types of properties (for example, a fluorescence spectrum and an absorption spectrum) and may be detected by different detection techniques or different detectors. As a result, it is possible to simultaneously determine the presence or absence of binding of biomarker 3 and the type of biomarker 3 based on the detected value at detection unit 9. In an example, the fluorescence spectra for the particles of the third particle group and those for the particles of the fourth particle group are configured so as not to overlap with the fluorescence spectra for the label properties of labeling portions 21az, 21cz, respectively.

### (5-3. Measurement Process According to Variation 2)

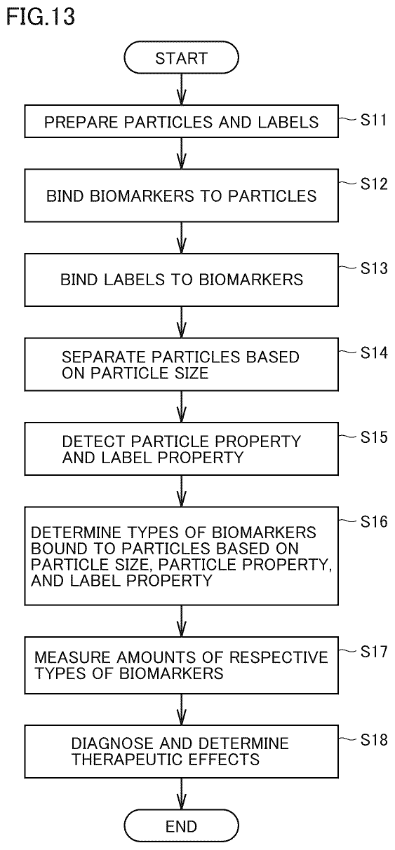

FIG. 13 is a flowchart illustrating a measurement process according to Variation 2. The steps illustrated in FIG. 13 are executed with the use of measurement system 100.

In S11, the user prepares particles 1z belonging to a third particle group and particles 1z belonging to a fourth particle group. The user also prepares labels 2z belonging to a third label group corresponding to the third particle group, and labels 2z belonging to a fourth label group corresponding to the fourth particle group. As described above, the particles belonging to the third particle group and the particles of the fourth particle group are different from each other in particle property.

In S12, with the use of pretreatment unit 71, the user mixes a specimen, particles 1z of the third particle group, and particles 1z of the fourth particle group to allow biomarkers 3 to specifically bind to particles 1z of the third particle group and particles 1z of the fourth particle group.

In S13, with the use of pretreatment unit 71, the user mixes the mixed solution prepared in S12, labels 2z of the third label group, and labels 2z of the fourth label group to allow biomarkers 3 to specifically bind to labels 2z of the third label group and labels 2z of the fourth label group.

In S14, processor 40 separates among particles 1z of the third particle group and particles 1z of the fourth particle group, based on the particle size.

In S15, processor 40 detects the particle property of each of particles 1z of the third particle group and particles 1z of the fourth particle group thus separated based on the particle size, as well as the label property of label 2z bound thereto via biomarker 3. For example, when detection unit 9 is a plurality of fluorescence detectors or multi-wavelength fluorescence detectors, processor 40 performs excitation light irradiation and detects the patterns of the fluorescence spectra thus exhibited.

In S16, based on the particle size, the particle property, and the label property, processor 40 determines the type of biomarker 3 bound to each of particles 1z of the third particle group and particles 1z of the fourth particle group.

In an example, based on the fluorescence spectrum patterns, processor 40 determines the type of biomarker 3 bound to each of particles 1z. More specifically, firstly based on a peak corresponding to label 2z in a fluorescence spectrum pattern which is a detected value, processor 40 determines whether biomarker 3 is bound to particle 1z. Then, based on peaks corresponding to the particle properties of particles 1z of the third particle group and particles 1z of the fourth particle group, processor 40 determines the type of particle 1z and determines the type of biomarker 3 corresponding thereto.

S17 to S18 correspond to S7 to S8 in FIG. 7, so the description thereof is not repeated.

With the measurement method according to Variation 2, it is possible to differentiate and detect multiple types of biomarkers 3 by using particles 1z having different combinations of particle size and particle property. As a result, it is possible to simultaneously measure multiple types of biomarkers 3 in a similar manner to the measurement method according to the embodiment.

### Aspects

As will be appreciated by those skilled in the art, the above-described example embodiments are specific examples of the below aspects.

(Item 1) A measurement method according to an aspect is a measurement method to measure a biomolecule contained in a specimen derived from a biological specimen, the measurement method comprising: preparing particles belonging to a first particle group and particles belonging to a second particle group; and preparing labels belonging to a first label group corresponding to the first particle group and labels belonging to a second label group corresponding to the second particle group.

The labels of the first label group and the labels of the second label group are different from each other in label property. The particle of the first particle group and the label of the first label group bind to each other via a biomolecule. The particle of the second particle group and the label of the second label group bind to each other via a biomolecule. Each of the first particle group and the second particle group includes a plurality of particle subgroups. In each of the first particle group and the second particle group, the plurality of particle subgroups are different from each other in particle size. Particles of the plurality of particle subgroups in the first particle group and particles of the plurality of particle subgroups in the second particle group have first binding portions, and the first binding portions for different particle subgroups are capable of specifically binding to different types of biomolecules.

The measurement method further comprises: mixing together the specimen, the particles of the first particle group and the particles of the second particle group, and the labels of the first label group and the labels of the second label group; allowing biomolecules to specifically bind to the particles of the first particle group and the particles of the second particle group as well as to the labels of the first label group and the labels of the second label group; separating among the particles of the first particle group and among the particles of the second particle group based on particle sizes; detecting label properties of the labels bound respectively via the biomolecules to the particles of the first particle group and the particles of the second particle group thus separated based on the particle sizes; and based on the particle sizes and the label properties, determining types of the biomolecules bound to the particles of the first particle group and the particles of the second particle group.

With the measurement method according to Item 1, it is possible to provide a technique for simultaneously measuring multiple types of biomolecules based on the difference in particle size and the difference in label property.

(Item 2) The measurement method according to Item 1 further comprises measuring amounts of respective types of the biomolecules based on results of determining the types of the biomolecules.

With the measurement method according to Item 2, it is possible to provide a technique for simultaneously measuring the amounts of respective types of biomolecules.

(Item 3) The measurement method according to Item 2 further comprises making a disease diagnosis or a therapeutic effect determination based on the amounts of the respective types of the biomolecules.

With the measurement method according to Item 3, it is possible to make a disease diagnosis or a therapeutic effect determination based on the amounts of respective types of biomolecules obtained by simultaneous measurement. In other words, it is possible to make a diagnosis based on multiple types of biomolecules, in an easy and simple manner.

(Item 4) In the measurement method according to any one of Items 1 to 3, the particle of each of the first particle group and the second particle group further includes a particle body, and the label of each of the first label group and the second label group includes a labeling portion, and a second binding portion capable of specifically binding to the biomolecule.

With the measurement method according to Item 4, it is possible to form label complexes each of which includes a particle via a first binding portion and a second binding portion. In addition, it is also possible to differentiate among the types of the label complexes based on the particle size mainly attributable to the particle body as well as on the label property of the labeling portion. As a result, it is possible to distinguish among the types of biomolecules corresponding to the label complexes.

(Item 5) In the measurement method according to Item 4, the labeling portion includes at least one of a fluorescent substance, a radioisotope, and a substance that exhibits a certain absorbance.

With the measurement method according to Item 5, it is possible to distinguish among the types of biomolecules based on the difference in the label property of the label substance.

(Item 6) In the measurement method according to Item 4 or 5, the biomolecule includes a protein, the first binding portion includes an antibody capable of binding to the protein, and the second binding portion includes an antibody capable of binding to the protein at a site different from a binding site to which the first binding portion binds.

In the measurement method according to Item 6, with antibody specificity, both the first binding portion and the second binding portion can bind to a certain type of protein, which is the biomolecule. Also, to the protein to which the particle is bound, the label can also bind.

(Item 7) In the measurement method according to any one of Items 1 to 6, the biomolecule includes at least one of a nucleic acid and a metabolite.

With the measurement method according to Item 7, not only proteins but also biomolecules belonging to nucleic acids and/or metabolites can be measured by the measurement method according to the present embodiment.

(Item 8) In the measurement method according to any one of Items 1 to 7, the particle of each of the first particle group and the second particle group includes at least one of an inorganic material and a resin material.

With the measurement method according to Item 8, the number of types of biomolecules simultaneously detectable can be increased, accuracy of biomolecule quantification is enhanced, and/or measurement can be performed relatively easily with accuracy.

(Item 9) In the measurement method according to Item 8, the inorganic material includes at least one of gold and silica.

With the measurement method according to Item 9, the number of types of biomolecules simultaneously detectable can be increased, and/or accuracy of biomolecule quantification is enhanced.

(Item 10) In the measurement method according to Item 8, the resin material includes polystyrene.

With the measurement method according to Item 10, because there are commercially available resin-material-based nanoparticles that are traceable and highly reliable, it is possible to perform measurement relatively easily with accuracy.

(Item 11) In the measurement method according to Items 1 to 10, the separating comprises separating the particles based on the particle sizes by using at least one of a centrifugal field flow fraction (FFF) method, an asymmetrical flow field flow fraction (AF4) method, and size exclusion chromatography.

In the measurement method according to Item 11, when a centrifugal FFF method is adopted, many types of biomolecules can be distinguished from each other and biomolecule quantification is performed with high accuracy. When an AF4 method is adopted, small, light-weight molecules can also be classified. When size exclusion chromatography is adopted, the apparatus can be configured at low cost.

(Item 12) In the measurement method according to any one of Items 1 to 11, in the detecting label properties, a plurality of fluorescence detectors or multi-wavelength fluorescence detectors are used.

In the measurement method according to Item 12, when a plurality of fluorescence detectors are used, highly sensitive detection of fluorescence is made possible, so accuracy of label property measurement is enhanced, and accuracy of biomolecule quantification is enhanced. When a multi-wavelength fluorescence detector is used, the number of detectors can be reduced, and thereby the cost for the detection unit can be reduced.

(Item 13) In the measurement method according to Item 12, the detecting comprises irradiating the particles of the first particle group and the second particle group mixed in a carrier, with excitation light. The preparing particles comprises preparing the particles of each of the first particle group and the second particle group as well as the carrier. The preparing the particles of each of the first particle group and the second particle group as well as the carrier comprises preparing the particles of each of the first particle group and the second particle group as well as the carrier where a difference between a refractive index of the particle of each of the first particle group and the second particle group and a refractive index of the carrier does not exceed a certain numerical value.

With the measurement method according to Item 13, it is possible to suppress the amount of particle-attributable scattering light at a certain amount or below. As a result, it is possible to set the lower limit to fluorescence detection low, and enhance detection sensitivity.

(Item 14) In the measurement method according to any one of Items 1 to 13, the detecting label properties further comprises correcting a detected value of the label property of the label bound to each of the particles of the first particle group and the particles of the second particle group.

With the measurement method according to Item 14, accuracy of biomolecule quantification in the measurement method is enhanced.

(Item 15) In the measurement method according to Item 14, the correcting comprises: preparing a first correction particle having a size corresponding to the particle size of each of the plurality of particle subgroups in each of the first particle group and the second particle group; for each particle size, detecting a label property of the first correction particle not bound with a labeling portion; and for each particle size, correcting the detected value of the label property of the labeling portion bound to each of the particles of the first particle group and the particles of the second particle group, based on a detected value of the label property of the first correction particle.

With the measurement method according to Item 15, it is possible to correct background while maintaining the number of types of biomolecules distinguishable from each other as high as possible.

(Item 16) In the measurement method according to Item 15, the detecting label properties of the first correction particle comprises: for each particle size, detecting the label property in a state where a biomolecule and a second binding portion are bound to the first correction particle including a first binding portion.

With the measurement method according to Item 16, it is possible to perform more accurate correction, taking into consideration the influence of the biomolecule and the second binding portion.

(Item 17) In the measurement method according to Item 14, the detecting comprises detecting a label property of a second correction particle having a particle size different from the particle size of each of the plurality of particle subgroups in each of the first particle group and the second particle group, in a state where the second correction particle is not bound with a labeling portion, and the correcting comprises correcting the detected value of the label property of the label bound to each of the particles of the first particle group and the particles of the second particle group, based on a detected value of the label property of the second correction particle.

With the measurement method according to Item 17, with no need for additional cost and time for separately measuring the background, it is possible to enhance the accuracy of biomolecule quantification.

(Item 18) In the measurement method according to Item 17, the correcting based on a detected value of the label property of the second correction particle comprises: correcting a detection result of the label property for binding to the particle of the first particle group and the particle of the second particle group, by using a correction coefficient calculated from a peak area of the label property of the second correction particle.

With the measurement method according to Item 18, by using the peak area for the second correction particle, it is possible to calculate the influence imposed on the peak area by a measurement-target particle that has a different particle size from the second correction particle.

(Item 19) A measurement method according to another aspect is a measurement method to measure a biomolecule contained in a specimen derived from a biological specimen, the measurement method comprising: preparing particles belonging to a third particle group and particles belonging to a fourth particle group; and preparing labels belonging to a third label group corresponding to the third particle group and labels belonging to a fourth label group corresponding to the fourth particle group.

The particles belonging to the third particle group and the particles of the fourth particle group are different from each other in particle property. The particle of the third particle group and the label of the third label group bind to each other via a biomolecule. The particle of the fourth particle group and the label of the fourth label group bind to each other via a biomolecule. Each of the third particle group and the fourth particle group includes a plurality of particle subgroups. In each of the third particle group and the fourth particle group, the plurality of particle subgroups are different from each other in particle size. Particles of the plurality of particle subgroups in the third particle group and particles of the plurality of particle subgroups in the fourth particle group have third binding portions, and the third binding portions for different particle subgroups are capable of specifically binding to different types of biomolecules.

The measurement method further comprises: mixing together the specimen, the particles of the third particle group and the particles of the fourth particle group, and the labels of the third label group and the labels of the fourth label group; allowing biomolecules to specifically bind to the particles of the third particle group and the particles of the fourth particle group as well as to the labels of the third label group and the labels of the fourth label group; separating among the particles of the third particle group and among the particles of the fourth particle group based on particle sizes; detecting particle properties of the particles of the third particle group and the particles of the fourth particle group thus separated based on the particle sizes, as well as label properties of the labels bound via the biomolecules to the particles; and based on the particle sizes, the particle properties, and the label properties, determining types of the biomolecules bound to the particles of the third particle group and the particles of the fourth particle group.

With the measurement method according to Item 19, it is possible to provide a technique for simultaneously measuring multiple types of biomolecules based on the difference in particle size and the difference in particle property.

(Item 20) In the measurement method according to Item 19, the particle property includes at least one of a fluorescence spectrum pattern, a radiation spectrum pattern, and an absorption spectrum pattern.

With the measurement method according to Item 20, it is possible to distinguish among different types of biomolecules based on the difference in the particle property.

(Item 21) In the measurement method according to Item 19 or 20, in the detecting, for each of the particles of the third particle group and the particles of the fourth particle group, a detected value of the particle property is distinguishable from a detected value of the label property.

With the measurement method according to Item 21, it is possible to simultaneously determine the presence or absence of binding of a biomolecule and the type of the biomolecule based on the detected value at the detection unit.

(Item 22) In the measurement method according to any one of Items 19 to 21, a detected value of the label property of the third label group is equivalent to a detected value of the label property of the fourth label group.

With the measurement method according to Item 22, it is possible to prepare the labeling portion in a less complicated manner.

(Item 23) A measurement method according to a yet another aspect is a measurement method to measure a biomolecule contained in a specimen derived from a biological specimen, the measurement method comprising: preparing particles belonging to a first particle group; and preparing labels belonging to a first label group corresponding to the first particle group.

The particle of the first particle group and the label of the first label group bind to each other via a biomolecule. The first particle group includes a plurality of particle subgroups. In the first particle group, the plurality of particle subgroups are different from each other in particle size. Particles of the plurality of particle subgroups in the first particle group have first binding portions, and the first binding portions for different particle subgroups are capable of specifically binding to different types of biomolecules.

The measurement method further comprises: mixing the specimen, the particles of the first particle group, and the labels of the first label group to allow biomolecules to specifically bind to the particles of the first particle group and the labels of the first label group; separating the particles of the first particle group based on particle sizes; detecting label properties of the labels bound respectively via the biomolecules to the particles of the first particle group thus separated based on the particle sizes; and based on the particle sizes and the label properties, determining types of the biomolecules bound to the particles of the first particle group.

With the measurement method according to Item 23, it is possible to bind particles of different particle sizes to multiple types of biomolecules contained in a specimen, respectively, and it is possible to provide a technique for simultaneously measuring the multiple types of biomolecules based on the difference in particle size.

It should be construed that embodiments disclosed herein are given by way of illustration in all respects, not by way of limitation. It is intended that the scope of the present invention is defined by claims, not by the above description, and encompasses all modifications and variations equivalent in meaning and scope to the claims.

### REFERENCE SIGNS LIST

1, 1a to 1d, 1z, 1az to 1dz particle, 1ar, 1br first correction particle, 1r second correction particle, 2, 2a to 2d, 2z, 2az to 2dz label, 3, 3a to 3d biomarker, 4 control apparatus, 5 measurement apparatus, 6 liquid unit, 8 separation unit, 9 detection unit, 11, 11a, 11b, 11az to 11dz particle body, 12, 12a to 12d, 12z, 12az to 1dz first binding portion, 21, 21a, 21c, 21az, 21cz labeling portion, 22, 22a to 22d second binding portion, 40 processor, 41 memory, 42 input unit, 43 display unit, 51, 52 analysis channel, 61 vessel, 62 pump, 71 pretreatment unit, 72 injection unit, 100 measurement system.

1. A measurement method to measure a biomolecule contained in a specimen derived from a biological specimen, the measurement method comprising:
preparing particles belonging to a first particle group and particles belonging to a second particle group; and
preparing labels belonging to a first label group corresponding to the first particle group and labels belonging to a second label group corresponding to the second particle group, wherein
the labels of the first label group and the labels of the second label group are different from each other in label property,
the particle of the first particle group and the label of the first label group bind to each other via a biomolecule,
the particle of the second particle group and the label of the second label group bind to each other via a biomolecule,
each of the first particle group and the second particle group includes a plurality of particle subgroups,
in each of the first particle group and the second particle group, the plurality of particle subgroups are different from each other in particle size,
particles of the plurality of particle subgroups in the first particle group and particles of the plurality of particle subgroups in the second particle group have first binding portions, and the first binding portions for different particle subgroups are capable of specifically binding to different types of biomolecules, and
the measurement method further comprises:
mixing together the specimen, the particles of the first particle group and the particles of the second particle group, and the labels of the first label group and the labels of the second label group;
allowing biomolecules to specifically bind to the particles of the first particle group and the particles of the second particle group as well as to the labels of the first label group and the labels of the second label group;
separating among the particles of the first particle group and among the particles of the second particle group based on particle sizes;
detecting label properties of the labels bound respectively via the biomolecules to the particles of the first particle group and the particles of the second particle group thus separated based on the particle sizes; and
based on the particle sizes and the label properties, determining types of the biomolecules bound to the particles of the first particle group and the particles of the second particle group.

2. The measurement method according to claim 1, further comprising measuring amounts of respective types of the biomolecules based on results of determining the types of the biomolecules.

3. The measurement method according to claim 2, further comprising making a disease diagnosis or a therapeutic effect determination based on the amounts of the respective types of the biomolecules.

4. The measurement method according to claim 1, wherein
the particle of each of the first particle group and the second particle group further includes a particle body, and
the label of each of the first label group and the second label group includes a labeling portion, and a second binding portion capable of specifically binding to the biomolecule.

5. The measurement method according to claim 4, wherein the labeling portion includes at least one of a fluorescent substance, a radioisotope, and a substance that exhibits a certain absorbance.

6. The measurement method according to claim 4, wherein
the biomolecule includes a protein,
the first binding portion includes an antibody capable of binding to the protein, and
the second binding portion includes an antibody capable of binding to the protein at a site different from a binding site to which the first binding portion binds.

7. The measurement method according to claim 1, wherein the biomolecule includes at least one of a nucleic acid and a metabolite.

8. The measurement method according to claim 1, wherein the particle of each of the first particle group and the second particle group includes at least one of an inorganic material and a resin material.

9. The measurement method according to claim 8, wherein the inorganic material includes at least one of gold and silica.

10. The measurement method according to claim 8, wherein the resin material includes polystyrene.

11. The measurement method according to claim 1, wherein the separating comprises separating the particles based on the particle sizes by using at least one of a centrifugal field flow fraction (FFF) method, an asymmetrical flow field flow fraction (AF4) method, and size exclusion chromatography.

12. The measurement method according to claim 1, wherein in the detecting label properties, a plurality of fluorescence detectors or multi-wavelength fluorescence detectors are used.

13. The measurement method according to claim 12, wherein
the detecting label properties comprises irradiating the particles of the first particle group and the second particle group mixed in a carrier, with excitation light,
the preparing particles comprises preparing the particles of each of the first particle group and the second particle group as well as the carrier, and
the preparing the particles of each of the first particle group and the second particle group as well as the carrier comprises preparing the particles of each of the first particle group and the second particle group as well as the carrier where a difference between a refractive index of the particle of each of the first particle group and the second particle group and a refractive index of the carrier does not exceed a certain numerical value.

14. The measurement method according to claim 1, wherein the detecting label properties further comprises correcting a detected value of the label property of the label bound to each of the particles of the first particle group and the particles of the second particle group.

15. The measurement method according to claim 14, wherein the correcting comprises:
preparing a first correction particle having a size corresponding to the particle size of each of the plurality of particle subgroups in each of the first particle group and the second particle group;
for each particle size, detecting a label property of the first correction particle not bound with a labeling portion; and
for each particle size, correcting the detected value of the label property of the labeling portion bound to each of the particles of the first particle group and the particles of the second particle group, based on a detected value of the label property of the first correction particle.

16. The measurement method according to claim 15, wherein the detecting a label property of the first correction particle comprises:
for each particle size, detecting the label property in a state where a biomolecule and a second binding portion are bound to the first correction particle including a first binding portion.

17. The measurement method according to claim 14, wherein
the detecting comprises detecting a label property of a second correction particle having a particle size different from the particle size of each of the plurality of particle subgroups in each of the first particle group and the second particle group, in a state where the second correction particle is not bound with a labeling portion, and
the correcting comprises correcting the detected value of the label property of the label bound to each of the particles of the first particle group and the particles of the second particle group, based on a detected value of the label property of the second correction particle.

18. The measurement method according to claim 17, wherein the correcting based on a detected value of the label property of the second correction particle comprises:
correcting a detection result of the label property of the label for binding to the particle of the first particle group and the particle of the second particle group, by using a correction coefficient calculated from a peak area of the label property of the second correction particle.

19. A measurement method to measure a biomolecule contained in a specimen derived from a biological specimen, the measurement method comprising:
preparing particles belonging to a third particle group and particles belonging to a fourth particle group; and
preparing labels belonging to a third label group corresponding to the third particle group and labels belonging to a fourth label group corresponding to the fourth particle group, wherein
the particles belonging to the third particle group and the particles of the fourth particle group are different from each other in particle property,
the particle of the third particle group and the label of the third label group bind to each other via a biomolecule,
the particle of the fourth particle group and the label of the fourth label group bind to each other via a biomolecule,
each of the third particle group and the fourth particle group includes a plurality of particle subgroups,
in each of the third particle group and the fourth particle group, the plurality of particle subgroups are different from each other in particle size,
particles of the plurality of particle subgroups in the third particle group and particles of the plurality of particle subgroups in the fourth particle group have third binding portions, and the third binding portions for different particle subgroups are capable of specifically binding to different types of biomolecules, and
the measurement method further comprises:
mixing together the specimen, the particles of the third particle group and the particles of the fourth particle group, and the labels of the third label group and the labels of the fourth label group;
allowing biomolecules to specifically bind to the particles of the third particle group and the particles of the fourth particle group as well as to the labels of the third label group and the labels of the fourth label group;
separating among the particles of the third particle group and among the particles of the fourth particle group based on particle sizes;
detecting particle properties of the particles of the third particle group and the particles of the fourth particle group thus separated based on the particle sizes, as well as label properties of the labels bound via the biomolecules to the particles; and
based on the particle sizes, the particle properties, and the label properties, determining types of the biomolecules bound to the particles of the third particle group and the particles of the fourth particle group.

21-21. (canceled)

22. The measurement method according to claim 19, wherein a detected value of the label property of the third label group is equivalent to a detected value of the label property of the fourth label group.

23. A measurement method to measure a biomolecule contained in a specimen derived from a biological specimen, the measurement method comprising:
preparing particles belonging to a first particle group; and
preparing labels belonging to a first label group corresponding to the first particle group, wherein
the particle of the first particle group and the label of the first label group bind to each other via a biomolecule,
the first particle group includes a plurality of particle subgroups,
in the first particle group, the plurality of particle subgroups are different from each other in particle size,
particles of the plurality of particle subgroups in the first particle group have first binding portions, and the first binding portions for different particle subgroups are capable of specifically binding to different types of biomolecules, and
the measurement method further comprises:
mixing the specimen, the particles of the first particle group, and the labels of the first label group to allow biomolecules to specifically bind to the particles of the first particle group and the labels of the first label group;
separating the particles of the first particle group based on particle sizes;
detecting label properties of the labels bound respectively via the biomolecules to the particles of the first particle group thus separated based on the particle sizes; and
based on the particle sizes and the label properties, determining types of the biomolecules bound to the particles of the first particle group.

24. The measurement method according to claim 19, wherein the particle property includes at least one of a fluorescence spectrum pattern, a radiation spectrum pattern, and an absorption spectrum pattern.

25. The measurement method according to claim 19, wherein in the detecting, for each of the particles of the third particle group and the particles of the fourth particle group, a detected value of the particle property is distinguishable from a detected value of the label property.
